SEQUENCE LISTING (60 sequences)

SEQ ID NO: 1 - protein, 11 aa, Artificial Sequence
  Synthetic SNP region
DVAGATFMAAG

SEQ ID NO: 2 - protein, 9 aa, Artificial Sequence
  Synthetic Myc tag
QKLISEEDL

SEQ ID NO: 3 - DNA, 1617 bp, Homo Sapiens
cctcacatgatccagtttaatcctcctcttctcccttcctgaagctgcacgctgcagtaagagcacagcagaaatgcagacaaaagggggccaaacatgggcgagaagggctctgttgctcggcatcctgtgggccactgcacatctgcctctctcagggacctccctgccccaacgtctcccaagggccacaggaaatagcacccaatgtgttatttctccatcatcggagtttcccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcacaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaataactggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggttgatattattaatagtgttatgcagaaaatatgaatggcaggga

SEQ ID NO: 4 - protein, 500 aa, Homo Sapiens
MQTKGGQTWARRALLLGILWATAHLPLSGTSLPQRLPRATGNSTQCVISPSSEFPEGFFTRQERRDGGIIIYFLIIVYMFMAISIVCDEYFLPSLEIISESLGLSQDVAGTTFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGG

SEQ ID NO: 5 - protein, 46 aa, Homo Sapiens
IVCDDFFVPSLEKICERLHLSEDVAGATFMAAGSSTPELFASVIGV

SEQ ID NO: 6 - protein, 46 aa, Homo Sapiens
IVCDDFFVPSLEKICERLHLSEDVAGATFMAAGSSAPELFTSVIGV

SEQ ID NO: 7 - protein, 46 aa, Homo Sapiens
IVCDEYFLPSLEIISESLGLSQDVAGTTFMAAGSSAPELVTAFLGV

SEQ ID NO: 8 - protein, 46 aa, Homo Sapiens
IVCDEFFVPSLTVITEKLGISDDVAGATFMAAGGSAPELFTSLIGV

SEQ ID NO: 9 - protein, 46 aa, Homo Sapiens
IVCDEYFVPALGVITDKLQISEDVAGATFMAAGGSAPELFTSLIGV

SEQ ID NO: 10 - DNA, 300 bp, Artificial Sequence
  Synthetic pIE1/153A (V4-) polylinker
ctaattcaagaggtgcgacgaagaagttgccgcgttggtagtagacggtatcgataagcttgatatcgaattcctgcagccctgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttcgagaagtcttacgaacttcccgacggtcaggtcatcaccatcggaaacgaaag

SEQ ID NO: 11 - DNA, 300 bp, Artificial Sequence
  Synthetic pIE1/153A (V4+) polylinker
ctaattcaagaggtgcgacgaagaagttgccgcgttggtagtagacggtatcgataagcttgatatcgaattcctgcagcccaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattagacgagaagtcttacgaacttcccgacggtcaggtcatcaccatcggaaacgaaag

SEQ ID NO: 12 - DNA, 22 bp, Artificial Sequence
  Synthetic Actin 1 primer
gacgaagaagttgccgcgttgg

SEQ ID NO: 13 - DNA, 22 bp, Artificial Sequence
  Synthetic Actin 3 primer
cgatggtgatgacctgaccgtc

SEQ ID NO: 14 - DNA, 1547 bp, Artificial Sequence
  Synthetic myc tagged human nckx5
ctcgagatgcagacaaaagggggccaaacatgggcgagaagggctctgttgctcggcatcctgtgggccactgcacatctgcctctctcagggacctccctgccccaacgtctcccaagggccacaggaaatagcacccaatgtgttatttctccatcatcggaacagaagctcatctcagaagaggatctggaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggttgagcggccgc

SEQ ID NO: 15 - protein, 510 aa, Artificial Sequence
  Synthetic myc tagged human nckx5
MQTKGGQTWARRALLLGILWATAHLPLSGTSLPQRLPRATGNSTQCVISPSSEQKLISEEDLEFPEGFFTRQERRDGGIIIYFLIIVYMFMAISIVCDEYFLPSLEIISESLGLSQDVAGATFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGG

SEQ ID NO: 16 - DNA, 1577 bp, Artificial Sequence
  Synthetic myc and id4 tagged human nckx5
ctcgagatgcagacaaaagggggccaaacatgggcgagaagggctctgttgctcggcatcctgtgggccactgcacatctgcctctctcagggacctccctgccccaacgtctcccaagggccacaggaaatagcacccaatgtgttatttctccatcatcggaacagaagctcatctcagaagaggatctggaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggtacagagacatcccaggtcgcaccagcatagtgagcggccgc

SEQ ID NO: 17 - protein, 519 aa, Artificial Sequence
  Synthetic myc and id4 tagged human nckx5
MQTKGGQTWARRALLLGILWATAHLPLSGTSLPQRLPRATGNSTQCVISPSSEQKLISEEDLEFPEGFFTRQERRDGGIIIYFLIIVYMFMAISIVCDEYFLPSLEIISESLGLSQDVAGATFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGGTETSQVAPA

SEQ ID NO: 18 - DNA, 1751 bp, Artificial Sequence
  Synthetic myc tagged nckx2/nckx5 chimera
ctcgagatggatctgcaacaaagcaccaccatcacttccctagagaaatggtgtttggatgagtcactgtctggctgcagaagacattacagtgtcaagaaaaaactgaagttaattcgagtcttaggccttttcatgggtctggtagccattagcactgtctcattttcaatcagtgccttttctgagacagatacacagagcacaggagaggccagtgttgtaagtggccctagggtagcacagggttaccatgaacaaaagctgatcagcgaagaagatctgggttaccatcagagaactctcttagatttaaatgacaagattctggattatactccacagccacctctttctaaggaaggcgagtctgagaatagtacagatcacgcccaagaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggttgagcggccgc

SEQ ID NO: 19 - protein, 578 aa, Artificial Sequence
  Synthetic myc tagged nckx2/nckx5 chimera
MDLQQSTTITSLEKWCLDESLSGCRRHYSVKKKLKLIRVLGLFMGLVAISTVSFSISAFSETDTQSTGEASVVSGPRVAQGYHEQKLISEEDLGYHQRTLLDLNDKILDYTPQPPLSKEGESENSTDHAQEFPEGFFTRQERRDGGIIIYFLIIVYMFMAISIVCDEYFLPSLEIISESLGLSQDVAGATFMAAGSSAPELVTAFLGVFITKGDIGISTILGSAIYNLLGICAACGLLSNTVSTLSCWPLFRDCAAYTISAAAVLGIIYDNQVYWYEGALLLLIYGLYVLVLCFDIKINQYIIKKCSPCCACLAKAMERSEQQPLMGWEDEGQPFIRRQSRTDSGIFYEDSGYSQLSISLHGLSQVSEDPPSVFNMPEADLKRIFWVLSLPIITLLFLTTPDCRKKFWKNYFVITFFMSAIWISAFTYILVWMVTITGETLEIPDTVMGLTLLAAGTSIPDTIASVLVARKGKGDMAMSNIVGSNVFDMLCLGIPWFIKTAFINGSAPAEVNSRGLTYITISLNISIIFLFLAVHFNGWKLDRKLGIVCLLSYLGLATLSVLYELGIIGNNKIRGCGG

SEQ ID NO: 20 - DNA, 1781 bp, Artificial Sequence
  Synthetic myc tagged human nckx2/nckx5 chimera with id4 tag
ctcgagatggatctgcaacaaagcaccaccatcacttccctagagaaatggtgtttggatgagtcactgtctggctgcagaagacattacagtgtcaagaaaaaactgaagttaattcgagtcttaggccttttcatgggtctggtagccattagcactgtctcattttcaatcagtgccttttctgagacagatacacagagcacaggagaggccagtgttgtaagtggccctagggtagcacagggttaccatgaacaaaagctgatcagcgaagaagatctgggttaccatcagagaactctcttagatttaaatgacaagattctggattatactccacagccacctctttctaaggaaggcgagtctgagaatagtacagatcacgcccaagaattccccgaagggtttttcacgagacaggagcgcagagatggaggcatcataatctatttcctaattatcgtttacatgttcatggccatatctattgtctgtgatgaatacttcctaccctccctggaaatcatcagtgaatcccttggattgtctcaggatgttgcaggcgcaactttcatggcagcgggcagttcagctcctgaattagttactgctttcctaggtgtatttatcacaaagggagatattggcattagcaccatccttggatctgcaatttataatctccttggcatctgtgctgcctgtggtttgctatctaatacggtctcaacactatcatgttggcccctattcagagactgtgcagcgtacacaattagtgcagcagcagttcttggtataatatatgacaaccaagtttactggtatgaaggggctttactgcttttgatatatggattgtatgttttggtgctgtgttttgacattaaaattaaccaatatattataaagaaatgcagtccttgctgcgcctgtcttgccaaagctatggagagaagtgaacaacagccactgatgggctgggaagatgaaggtcaaccattcattcgtcggcaatcaagaactgatagtggaatattttatgaagattctggctactctcagctctctataagtttacatggccttagtcaggtttctgaagatccaccaagtgttttcaacatgcctgaagcagacttaaaaagaattttttgggtattatcccttcctattattacattactttttctaaccacaccagattgtagaaaaaagttttggaaaaactactttgtgataacctttttcatgtctgcaatatggatatccgcatttacatatatcctggtttggatggtcacaattacaggggaaacactagaaattcccgatacagtaatgggccttactttattagcagcaggaacaagcataccagacacaattgcaagtgtgttggttgcaagaaaagggaaaggagatatggctatgtctaacatcgtgggatccaatgtgtttgatatgttgtgccttggtattccatggtttattaaaactgcatttataaatggatcagctcctgcagaagtaaacagcagaggactaacttacataaccatctctctcaacatttcaattatttttctttttttagcagttcacttcaatggctggaaactagacagaaagttgggaatagtctgcctattatcatacttggggcttgctacattatcagttctatatgaacttggaattattggaaataataaaataaggggctgtggaggtacagagacatcccaggtcgcaccagcatagtgagcggccgc

SEQ ID NO: 21 - protein, 587 aa, Artificial Sequence [sequence omitted]

SEQ ID NO: 22 - RNA, 25 nt, Artificial Sequence
  Synthetic RNAi duplex 370 antisense oligo
guccuacaacguccacguugaaagu

SEQ ID NO: 23 - RNA, 25 nt, Artificial Sequence
  Synthetic RNAi duplex 370 sense oligo
caggauguugcaggugcaacuuuca

SEQ ID NO: 24 - RNA, 25 nt, Artificial Sequence
  Synthetic RNAi duplex 370 scrambled control antisense oligo
gucaacacugcgcacaguuaucagu

SEQ ID NO: 25 - RNA, 25 nt, Artificial Sequence
  Synthetic RNAi duplex 370 scrambled control sense oligo
caguugugacgcgugucaauaguca

SEQ ID NO: 26 - RNA, 25 nt, Artificial Sequence
  Synthetic RNAi duplex 762 antisense oligo
gucucgauaccuccucucuuaacuu

SEQ ID NO: 27 - RNA, 25 nt, Artificial Sequence
  Synthetic RNAi duplex 762 sense oligo
cagagcuauggaggagagaauugaa

SEQ ID NO: 28 - RNA, 25 nt, Artificial Sequence
  Synthetic RNAi duplex 762 scrambled control antisense oligo
gucauagcuccucucauucacucuu

SEQ ID NO: 29 - RNA, 25 nt, Artificial Sequence
  Synthetic RNAi duplex 762 scrambled control sense oligo
caguaucgaggagaguaagugagaa

SEQ ID NO: 30 - RNA, 25 nt, Artificial Sequence
  Synthetic RNAi duplex 1265 antisense oligo
agcacccuagguuacacaaacuaua

SEQ ID NO: 31 - RNA, 25 nt, Artificial Sequence
  Synthetic RNAi duplex 1265 sense oligo
ucgugggauccaauguguuugauau

SEQ ID NO: 32 - RNA, 25 nt, Artificial Sequence
  Synthetic RNAi duplex 265 antisense oligo
ccauagcauuagaugaaggaguaau

SEQ ID NO: 33 - RNA, 25 nt, Artificial Sequence
  Synthetic RNAi duplex 265 sense oligo
gguaucguaaucuacuuccucauua

SEQ ID NO: 34 - RNA, 25 nt, Artificial Sequence
  Synthetic RNAi duplex 567 antisense oligo
ggagaaggcucugacacgucacaua

SEQ ID NO: 35 - RNA, 25 nt, Artificial Sequence
  Synthetic RNAi duplex 567 sense oligo
ccucuuccgagacugugcaguguau

SEQ ID NO: 36 - DNA, 18 bp, Artificial Sequence
  Synthetic SLC24A1 primer forward
tctgcacaacagcaccat

SEQ ID NO: 37 - DNA, 20 bp, Artificial Sequence
  Synthetic SLC24A1 primer reverse
ctctcctcctccttctcctt

SEQ ID NO: 38 - DNA, 19 bp, Artificial Sequence
  Synthetic SLC24A2 primer forward
atgatacacacccttgacc

SEQ ID NO: 39 - DNA, 21 bp, Artificial Sequence
  Synthetic SLC24A2 primer reverse
ccttttctctgaacctccctt

SEQ ID NO: 40 - DNA, 22 bp, Artificial Sequence
  Synthetic SLC24A3 primer forward
cgtcttatacttcactgtaccc

SEQ ID NO: 41 - DNA, 21 bp, Artificial Sequence
  Synthetic SLC24A3 primer reverse
aaccaatgattgtgaccatcc

SEQ ID NO: 42 - DNA, 18 bp, Artificial Sequence
  Synthetic SLC24A4 primer forward
gacacagacagccaagaa

SEQ ID NO: 43 - DNA, 24 bp, Artificial Sequence
  Synthetic SLC24A4 primer reverse
gcatagaacatatacagagcacca

SEQ ID NO: 44 - DNA, 22 bp, Artificial Sequence
  Synthetic SLC24A5 primer forward
gagatggaggcatcataatcta

SEQ ID NO: 45 - DNA, 22 bp, Artificial Sequence
  Synthetic SLC24A5 primer reverse
cctgagacaatccaagggattc

SEQ ID NO: 46 - DNA, 17 bp, Artificial Sequence
  Synthetic SLC24A6 primer forward
aggcttcactggctctt

SEQ ID NO: 47 - DNA, 20 bp, Artificial Sequence
  Synthetic SLC24A6 primer reverse
aggcatctccaatgctgttc

SEQ ID NO: 48 - DNA, 18 bp, Artificial Sequence
  Synthetic GAPDH primer forward
ggacctgacctgccgtct

SEQ ID NO: 49 - DNA, 18 bp, Artificial Sequence
  Synthetic GAPDH primer reverse
tagcccaggatgcccttg

SEQ ID NO: 50 - protein, 10 aa, Artificial Sequence
  Synthetic NCKX5 C-terminal peptide
GNNKIRGCGG

SEQ ID NO: 51 - DNA, 25 bp, Artificial Sequence
  Synthetic RNAi duplex 185 sense oligo
agggccacaggaaatagcacccaat

SEQ ID NO: 52 - DNA, 25 bp, Artificial Sequence
  Synthetic RNAi duplex 185 antisense oligo
attgggtgctatttcctgtggccct

SEQ ID NO: 53 - DNA, 25 bp, Artificial Sequence
  Synthetic RNAi duplex 260 sense oligo
gagcgcagagatggaggcatcataa

SEQ ID NO: 54 - DNA, 25 bp, Artificial Sequence
  Synthetic RNAi duplex 260 antisense oligo
ttatgatgcctccatctctgcgctc

SEQ ID NO: 55 - DNA, 25 bp, Artificial Sequence
  Synthetic RNAi duplex 301 sense oligo
cgtttacatgttcatggccatatct

SEQ ID NO: 56 - DNA, 25 bp, Artificial Sequence
  Synthetic RNAi duplex 301 antisense oligo
agatatggccatgaacatgtaaacg

SEQ ID NO: 57 - DNA, 25 bp, Artificial Sequence
  Synthetic RNAi duplex 492 sense oligo
gcaccatccttggatctgcaattta

SEQ ID NO: 58 - DNA, 25 bp, Artificial Sequence
  Synthetic RNAi duplex 492 antisense oligo
taaattgcagatccaaggatggtgc

SEQ ID NO: 59 - DNA, 25 bp, Artificial Sequence
  Synthetic RNAi duplex 1110 sense oligo
ccgcatttacatatatcctggtttg

SEQ ID NO: 60 - DNA, 25 bp, Artificial Sequence
  Synthetic RNAi duplex 1110 antisense oligo
caaaccaggatatatgtaaatgcgg

(19) United States
(12) Patent Application Publication
(10) Pub. No.: US 2010/0249373 A1
(43) Pub. Date: Sep. 30, 2010

(54) SCREENING METHODS

(76) Inventors: Richard Martin Ogborne, Rushden (GB); Paul Petrus Marius Schnetkamp, Calgary (CA)

Correspondence Address: MORGAN LEWIS & BOCKIUS LLP, 1111 PENNSYLVANIA AVENUE NW, WASHINGTON, DC, 20004, US

(21) Appl. No.: 12/743,519

(22) PCT Filed: Nov. 11, 2008
(86) PCT No.: PCT/EP2008/065330

§ 371 Date: May 18, 2010

(30) Foreign Application Priority Data
Nov. 27, 2007 (EP) 07121613.9

(51) Int. Cl. C12Q 1/02; A61K 49/00; C07K 5/06; C07K 5/08; C07H 19/00

(52) U.S. Cl. 530/331; 435/29; 424/9.2; 562/553; 536/22.1

(57) ABSTRACT

A method of identifying a compound that modulates NCKX-mediated calcium ion exchange across a cell membrane, the method comprising the steps of providing an adherent cell, the cell membrane of which comprises a NCKX polypeptide or a functionally equivalent variant thereof, exposing the cell in suspension to a fluorescent calcium-sensitive dye, thereby causing intracellular uptake of the dye, settling the cell onto a solid support without allowing the cell to adhere to the solid support, exposing the cell to a test compound, exposing the cell to calcium and potassium ions, and measuring fluorescence from the intracellular calcium-sensitive dye after exposure of the cell to the test compound and the calcium and potassium ions, thereby to determine the rate and/or amount of calcium ion exchange across the cell membrane in the presence of the test compound.

The invention relates to screening methods, and in particular to methods of identifying compounds having activity in modulating NCKX-mediated ion exchange across a membrane.

The NCKX (Na/Ca—K exchanger) family of ion exchange proteins mediates sodium (Na⁺) and potassium (K⁺) dependent calcium ion (Ca²⁺) movement across a membrane. The NCKX exchangers can operate either in a forward (Ca²⁺ exit) mode or in a reverse (Ca²⁺ entry) mode, depending on the Na⁺, K⁺ and Ca²⁺ gradients and the potential across the plasma membrane. Six members of this family have been identified to date, NCKX1-6 (Tsoi et al (1998) J Biol Chem 273: 4155-4162; Blaustein & Lederer (1999) Physiol Rev 79: 763-854; Kraev et al (2001) J Biol Chem 276: 23161-23172; Lytton et al (2002) Ann NY Acad Sci 976: 382-393; Li et al (2002) J Biol Chem 277: 48410-48417; Cai & Lytton (2004) J Biol Chem 279: 5867-5876). NCKX1 is expressed in retinal rod photoreceptors, NCKX2 is expressed in brain neurons and cone photoreceptors, NCKX3 and NCKX4 are expressed in brain and in other tissues, including aorta, uterus, and intestine, which are rich in smooth muscle cells, and NCKX5 is expressed in brain, skin and retinal pigment epithelium (RPE), where it is thought to present on the melanosome membrane and not the plasma membrane (Lamason et al (2005) Science 310: 1782-1786; Lytton (2007) Biochem J 406: 365-382).

The NCKX proteins have an important physiological role in retinal rod photoreceptors, brain, spermatozoa, mast cells, platelets and smooth muscle cells. For example, NCKX1 and NCKX2 exhibit calcium exchange functions in human photoreceptors and bovine heart muscle (Winkfein et al (2003) Biochemistry 42: 543-552; Schnetkamp (1996) Biochem. Cell. Biol. 74: 535-539) and NCKX3 and NCKX4 have been demonstrated to exhibit calcium exchange function in arterial smooth muscle (Dong et al (2006) American Journal Physiol. Heart Circ. Physiol 291: 1226-1235). We therefore consider that modulators of NCKX-mediated ion exchange may be therapeutically beneficial in diseases or conditions where there is inappropriate or undesirable levels of NCKX expression or activity, such as diseases or disorders of the brain, retina and vascular smooth muscle cells.

NCKX5 (also known as SLC24A5) has been found to influence pigmentation in zebrafish and mammals. In the zebrafish, a mutation in NCKX5 exhibits a reduction in pigmentation associated with the “golden” mutation. The human gene also has a role in skin pigmentation; human NCKX5 mRNA rescued melanin pigmentation when injected into golden zebrafish embryos, demonstrating functional conservation of mammalian and fish polypeptides (Lamason et al, 2005). Using quantitative RT-PCR, Lamason et al (2005) examined NCKX5 expression in normal mouse tissues and in the B16 melanoma cell line. NCKX5 expression varied 1.000-fold between tissues, with concentrations in skin and eye at least 10-fold higher than in other tissues. Mouse melanoma showed approximately 100-fold greater expression of Slc24a5 (Nckx5) compared with normal skin and eye. NCKX5 expression has also been detected in human retinal pigment epithelium (Genbank Accession No DQ665306).

The skin is the largest organ in the body and has roles in thermoregulation, protection from physical and chemical injury, protection from infection and manufacture of Vitamin D. There is a broad range of skin colours which can be correlated to climates, continents and cultures. Predominantly darker skins are located in hotter climates closer to the equator and are thought to provide protection against UV radiation and the heat. Lighter skins are found in cooler areas where there is less need for UV protection and are also associated with increased vitamin D production. The principal pigments responsible for skin colour are carotene, haemoglobin and in particular melanin. Melanin is composed of two major sub-types, the darker eumelanin and lighter pheomelanin. Melanin is synthesised by melanocytes, and combined with other proteins into granules which are then redistributed to keratinocytes. The amount of melanin is influenced by exposure to UV radiation (tanning) and a darker skin can therefore be achieved by increasing the amount of melanin in the skin. The genetic basis for constitutive and induced skin pigmentation is not yet fully understood and it is hypothesised that a plurality of genes are involved in pigmentation. Different variants of these genes influence the skin colour phenotype of an individual before external factors such as sunlight influence skin colour.

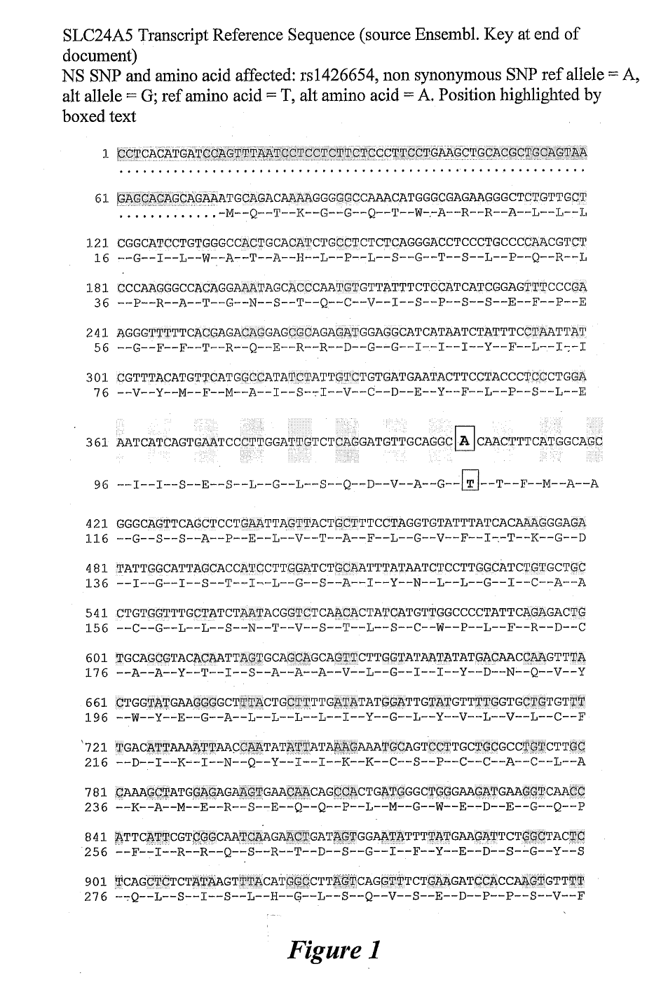

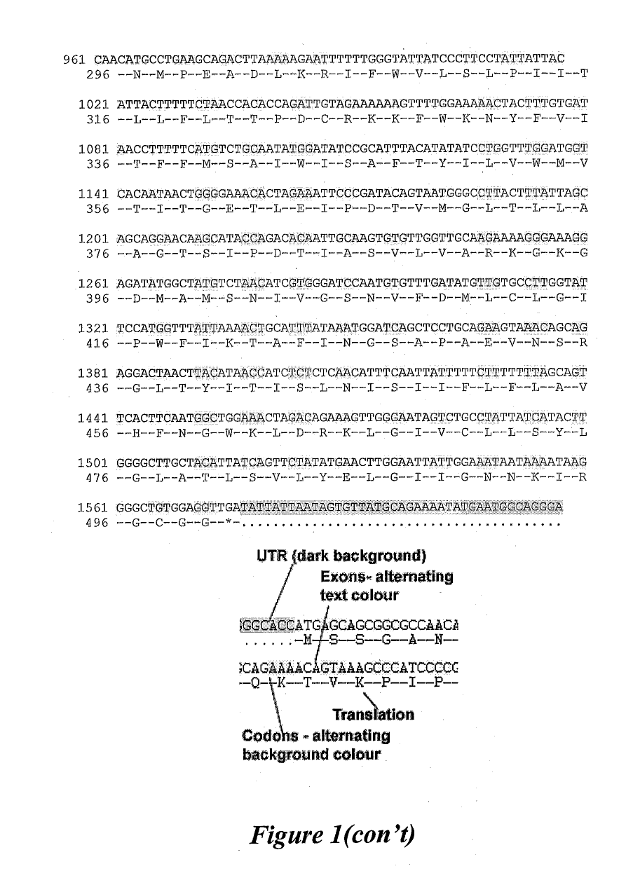

We have shown that NCKX5 is associated with sodium-potassium/calcium exchange function in melanocytes and that this function is closely correlated to skin pigmentation. In addition, we have shown that NCKX5 exists in two allelic forms at amino acid 111 due to a single nucleotide polymorphism (SNP). The Ala111 version is correlated with increased calcium exchange and is found predominantly in dark skin. The Thr111 version is closely correlated to decreased calcium exchange and is found predominantly in lighter skin (see Examples 1 and 2). The sequence of human NCKX5 including the position of the SNP is shown in FIG. 1. Since NCKX5 is involved in pigmentation, we consider that modulators of NCKX5-mediated ion exchange will modulate pigmentation.

There is thus a need in the art for simple, rapid and reproducible high-throughput methods of screening compounds for the ability to modulate NCKX-mediated ion exchange.

In a first aspect of the invention there is provided a method of identifying a compound that modulates NCKX-mediated calcium ion exchange across a cell membrane, the method comprising the steps of:
(a) providing an adherent cell, the cell membrane of which comprises a NCKX polypeptide or a functionally equivalent variant thereof;
(b) exposing the cell in suspension to a fluorescent calcium-sensitive dye, thereby causing intracellular uptake of the dye;
(c) settling the cell onto a solid support without allowing the cell to adhere to the solid support;
(d) exposing the cell to a test compound;
(e) exposing the cell to calcium ions (Ca²⁺) and potassium ions (K⁺); and
(f) measuring the fluorescence of the intracellular calcium-sensitive dye after exposure of the cell to the test compound and the calcium and potassium ions, thereby to determine the rate and/or amount of calcium ion exchange across the cell membrane in the presence of the test compound.

Steps (a) and (b) are performed prior to steps (c) to (f). It is preferred that step (c) is performed before steps (d) to (f), although in an alternative embodiment step (c) may be performed after step (d). In the embodiment when steps (d) and (e) are performed after step (c), steps (d) and (e) may be performed in either order, or substantially simultaneously.

Preferably, steps (a) to (f) are performed in that order.

By “NCKX-mediated” calcium ion exchange across a cell membrane, we mean that the calcium ion exchange is mediated by an NCKX polypeptide. The NCKX polypeptide may be derived from any species, in particular mammals and most preferably humans. Examples of NCKX polypeptides are NCKX1 (cDNA accession no. NM_{—}004727, protein accession no. NP_{—}004718); NCKX2 (cDNA accession no. NM_{—}020344, protein accession no. NP_{—}065077); NCKX3 (cDNA accession no. NM_{—}020689, protein accession no. NP_{—}065740); NCKX4 (cDNA accession nos. NM_{—}153648, NM_{—}153646, NM_{—}153647; protein accession nos. NP_{—}705934, NP_{—}705932, NP 705933); NCKX5 (see FIG. 1 for sequence, and cDNA accession nos. NM_{—}205850, XM_{—}208771, protein accession nos. NP_{—}995322, XP_{—}208771) and NCKX6 (cDNA accession no. NM_{—}024959, protein accession no. NP_{—}079235) (see, Cai & Lytton (2004) Mol Biol & Evolution 21(9): 1692-1703 and Schnetkamp (2004) Pflugers Arch—Eur J Physiol 447: 683-688).

In one preferred embodiment, the NCKX polypeptide is NCKX5, most preferably human NCKX5. NCKX5 is also known as SLC24A5.

In another preferred embodiment, the NCKX polypeptide is NCKX2, most preferably human NCKX2.

In other embodiments, the NCKX polypeptide may be NCKX1, NCKX3, or NCKX4, preferably human NCKX1, NCKX3, or NCKX4.

NCKX6 is least preferred.

In an embodiment, the NCKX polypeptide may be located in the cell membrane naturally, i.e. the cell is one that naturally expresses that NCKX molecule in the cell membrane. For example, the cell may be a retinal rod photoreceptor cell that naturally expresses NCKX1 in the cell membrane, or the cell may be a brain neuron or cone photoreceptor cell that naturally expresses NCKX2 in the cell membrane, or the cell may be a brain cell or a smooth muscle cell derived from tissues such as aorta, uterus and intestine which expresses NCKX3 or NCKX4 in the cell membrane, or a cell line derived therefrom.

By the term “cell membrane”, also known as the plasma membrane, we mean the external lipid bilayer structure at the surface of the cell. We do not include the internal membranes surrounding intracellular compartments.

In an alternative embodiment, the NCKX polypeptide or the functionally equivalent variant thereof may have been recombinantly targeted to the cell membrane. Typically, a nucleic acid molecule encoding the NCKX polypeptide, or the functionally equivalent variant thereof, is transfected into a cell in which it is not normally expressed, for example a cell from a species which does not have an equivalent polypeptide, under the control of a suitable promoter, usually in the form of an expression vector containing the nucleic acid molecule, thereby to direct expression of the NCKX polypeptide or variant to the cell membrane.

In one embodiment, the full-length NCKX polypeptide may be recombinantly targeted to the cell membrane. Thus, for example, we have demonstrated that, following transfection, full length NCKX5 is expressed in the cell membrane of High Five™ insect cells (data not shown).

Alternatively, the cell may have been transfected with a functionally equivalent variant of an NCKX polypeptide.

By a “functionally equivalent variant” of an NCKX polypeptide we mean a variant of the NCKX polypeptide whose ability to act as an ion exchanger has not significantly been changed. “Significantly” in this context means that one skilled in the art would say that the properties of the variant may still be different but would not be unobvious over the ones of the original protein. For example, a functionally equivalent variant of an NCKX polypeptide typically has at least 50% of the ion-exchange ability of the full-length native (i.e. non-variant) protein having the sequence specified in the Genbank Accession Nos. mentioned above. Preferably, the variant has at least 60%, 70% or 80% of the ion-exchange ability, and more preferably at least 90%, or 95%, or at least 99% of the of the ion-exchange ability of the full-length native protein. Most preferably, the variant has 100% or more of the ion-exchange ability of the full-length native protein.

Methods of determining the ability of a polypeptide, such as a variant NCKX polypeptide, to act as an ion-exchanger are well known in the art, and include those described herein. Typically, a cuvette method or radioactive calcium method would be used (Szerencsei et al (2000) J Biol Chem 275: 669-676; Winkfein et al (2003) Biochemistry 42: 543-52; Kang et al (2005a) J Biol Chem 280: 6823-33).

By a “functionally equivalent variant” of an NCKX polypeptide we include a sequence variant of the NCKX polypeptide wherein at one or more positions there have been amino acid insertions, deletions, or substitutions, either conservative or non-conservative, provided that such changes result in a sequence variant whose ability to act as an ion exchanger has not significantly been changed, as defined above. By “conservative substitutions” is intended combinations such as Gly, Ala; Val, Ile, Leu; Asp, Glu; Asn, Gln; Ser, Thr; Lys, Arg; and Phe, Tyr. Typically, the sequence variant has at least 65% sequence identity to the full-length native NCKX protein sequence. In one embodiment, the variant has at least 70%, more preferably at least 80%, yet more preferably at least 90%, and still more preferably at least 95% or at least 98% or at least 99% sequence identity to the full-length native NCKX protein sequence. The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., (1994) Nucleic Acids Res 22, 4673-80). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

By a “functionally equivalent variant” of an NCKX polypeptide we also include a fusion polypeptide in which the NCKX polypeptide, or sequence variant thereof, has been operatively linked to another polypeptide. In one embodiment, a fusion protein comprises at least one biologically active portion of the NCKX polypeptide. In another embodiment, a fusion protein comprises at least two biologically active portions of the NCKX polypeptide. Within the fusion protein, the term “operatively linked” is intended to indicate that the NCKX polypeptide and the other polypeptide are fused in-frame to each other. The other polypeptide can be fused to the N-terminus or C-terminus, or to the middle of the NCKX polypeptide. It is preferred that the ability of the functionally equivalent fusion polypeptide to act as an ion exchanger has not significantly been changed, as defined above.

Advantageously, the NCKX polypeptide is targeted to the cell membrane in the form of a fusion protein in which the NCKX polypeptide is linked to a leader sequence and/or tag that targets polypeptides to, and for inclusion in, the cell membrane. Conveniently, the leader sequence may be derived from an NCKX protein such as NCKX2 or NCKX4, yeast a mating factor, NCX proteins, TGFbeta, haemagglutinin or a viral surface protein. In an embodiment, the leader sequence is the N terminal sequence of human NCKX2 (amino acids 1 to 120).

It is appreciated that the NCKX polypeptide which is linked to the leader sequence may not comprise the full length NCKX polypeptide as specified in the Genbank Accession Nos. mentioned above. For example, the NCKX polypeptide may be shortened from the N-terminal end.

In specific embodiments, the fusion protein may comprise the N-terminus of NCKX2 (residues 1-120) and the C-terminus of NCKX5 (residues 151 to end); or the N-terminus of NCKX4 (residues 1-200) and the C-terminus of NCKX5 (residues 151 to end). Alternatively the fusion protein may comprise the C terminus of NCKX2 (11 amino acids) instead of the final C-terminal 11 amino acids of NCKX5.

Alternatively, the functionally equivalent variant NCKX polypeptide may be targeted to the cell membrane by deletion of internal compartment retention signals, such as ArgArg motifs present in NCKX3, 4 and 5.

It is appreciated that residues critical for Na/K/Ca binding to the exchanger are retained in functionally equivalent variant NCKX polypeptide. Critical residues include those described in Kang et al (2005a); Kang et al (2005b) J Biol. Chem. 280: 6834-9; Winkfein et al (2003); and Visser et al (2007). J Biol Chem 282(7): 4453-62. These include Thr-551 in NCKX2.

By a “functionally equivalent variant” of an NCKX polypeptide we also include a derivative of an NCKX polypeptide in which the native NCKX polypeptide has been modified. The derivative may have been modified by the addition of one or more naturally or non-naturally occurring amino acids or other molecules, e.g. to facilitate coupling of the polypeptide to another peptide, to a large carrier protein or to a solid support, its insertion into a membrane (e.g. the amino acids tyrosine, lysine, glutamic acid, aspartic acid, cysteine and derivatives thereof, NH₂-acetyl groups or COOH-terminal amido groups, amongst others), for subsequent detection (e.g. a Myc tag), transmembrane domain deletions, cytoplasmic loop deletions, or removal of N- or O-linked glycosylation sites. Preferably, the ability of the functionally equivalent derivative to act as an ion exchanger has not significantly been changed, as defined above.

By a “functionally equivalent variant” of an NCKX polypeptide we also include a fragment of the full length native polypeptide. To be active, the polypeptide fragment must have sufficient length to display biological activity. Thus, by a “fragment”, we include a stretch of at least 17 consecutive amino acid residues found in the full length native sequence, and which retains at least 50% of the ion-exchange activity of the full length native polypeptide as discussed above. More preferably, the fragment contains at least 50, or at least 100, or at least 200, or at least 300, or at least 400, or at least 450 consecutive residues found in the full length native sequence.

It will be appreciated that by “functionally equivalent variants” of an NCKX polypeptide we also include combinations of sequence variants, fusions, derivatives and fragments, in which the ability of the functionally equivalent variant to act as an ion exchanger has not significantly been changed, as defined above.

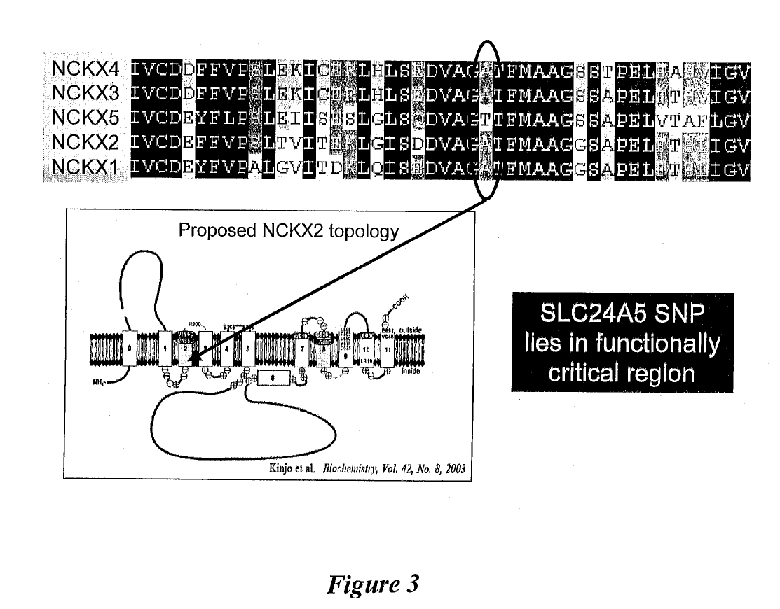

The SNP at the codon for amino acid residue 111 of human NCKX5 can code for either Ala or Thr (DVAGA/TTFMAAG) (see FIG. 3). The residue Thr is significantly associated with individuals having a light skin colour whereas Ala is associated with dark skin. Other NCKX molecule are high homologous to NCKX5 at the positions equivalent to this SNP in NCKX5 (FIG. 3). For example, the equivalent position in NCKX2 is residue 177. We have found that by substituting the naturally-occurring residue Ala with the amino acid Thr at this position in NCKX2, the ion-exchanger activity of the polypeptide is significantly reduced. Thus, in an embodiment, the NCKX polypeptide or functionally equivalent variant thereof may have Ala at the position equivalent to residue 111 of NCKX5 (which is residue 177 of NCKX2). In an alternative embodiment, the NCKX polypeptide or functionally equivalent variant thereof may have Thr at the position equivalent to residue 111 of NCKX5 (residue 177 of NCKX2).

Since the residue Thr at this SNP position is associated with individuals having a light skin colour and reduced ion exchanger activity, it may be advantageous to use an NCKX polypeptide, or functionally equivalent variant thereof, having Thr at this position when it is desired to identify a compound that may increase ion exchanger activity or increase pigmentation.

Conversely, since the residue Ala at the SNP position is associated with individuals having a darker skin colour and increased ion exchanger activity, it may be advantageous to use an NCKX polypeptide, or functionally equivalent variant thereof, having Ala at this position when it is desired to identify a compound that may decrease ion exchanger activity or decrease pigmentation.

Methods for producing nucleic acid molecules that encode an NCKX polypeptide, such as such as a full length NCKX polypeptide, or functionally equivalent variant thereof, under the control of a suitable promoter, typically in the form of an expression vector containing the nucleic acid molecule, are very well known in the art. Similarly, methods for transfecting a cell with nucleic acid molecules and expression vectors so that the encoded proteins are expressed in the cell and targeted to the cell membrane, are very well known in the art. For example, suitable techniques for cloning, manipulation, modification and expression of nucleic acids, and the culture, transformation and analysis of cells, are described in Sambrook et al (2001) “Molecular Cloning, a Laboratory Manual”, 3^{rd} edition, Sambrook et al (Eds.), Cold Spring Harbor Laboratory Press, Cold Spring Harbor, N.Y., USA; Freshney (2000) “Culture of Animal Cells: A Manual of Basic Technique”, 4th edition, Freshney (Ed.), Wiley-Liss, Inc., NY, USA; and Ausubel et al (2002) “Short Protocols in Molecular Biology”, Ausubel et al (Eds.), John Wiley and Sons, Inc., NY, USA.

By an “adherent cell” we mean that is one that grows in an adherent fashion in cell culture, as is very well known in the art.

Many suitable adherent cells for carrying out the screening method are known in the art and include mammalian cells such as Hamster Embryonic Kidney (HEK) cells, Chinese hamster ovary (CHO) cells available from the ATCC as CCL61, NIH Swiss mouse embryo cells NIH/3T3 available from the ATCC as CRL 1658, and monkey kidney-derived COS-1 cells available from the ATCC as CRL 1650. Possible insect cells include High Five™ and Sf9 cells which can be transfected with baculovirus expression vectors. Other possible cells include yeast cells, dictyostelium cells, tobacco plant cells, cells of the p53 deficient cell line H1299.

In an embodiment, the adherent cell is not a human embryonic stem cell.

Typically, prior to step (b), the adherent cell is trypsinised, thereby to bring the cell into suspension.

In step (c) the cells are settled onto the solid support, which is typically a cell culture chamber or well. It is preferred that the method is carried out in a multi-well format, for example in 96- or 384-well plates. It may also be preferred if the assay plate or well is treated to facilitate cell binding, for example the well may be coated with a high binding capacity polystyrene resin.

Most preferably, step (c) of settling the cell onto a solid support comprises centrifuging the cell so that it settles at the bottom of the cell culture chamber or well. Suitable centrifugation conditions are 300 g for 60 sec.

In step (c), the cell is not allowed to adhere to the solid support. This provides the advantages of speed, ease of use and reproducibility. Thus, typically, steps (d) to (f) are carried out within 6 hours of step (c), preferably within 4 hours, 3 hours, 2 hours, or 1 hour of step (c), and more preferably within 30 minutes, 15 minutes, 10 minutes or 5 minutes of step (c). When step (c) comprises centrifuging the cell, steps (d) to (f) are carried out within 6 hours, or 4 hours, or 3 hours, or 2 hours, or within 1 hour, and more preferably within 30 minutes, 15 minutes, 10 minutes or 5 minutes of the centrifugation in step (c). In the event that step (d) is performed before step (c), steps (e) and (f) are carried out within 6 hours of step (c), preferably within 4 hours, 3 hours, 2 hours, or 1 hour of step (c), and more preferably within 30 minutes, 15 minutes, 10 minutes or 5 minutes of step (c), for example within these times from the centrifugation in step (c).

It is well known in the art, changes in cytosolic calcium ions can be detected, for example, by devices such as FLIPR™ (Molecular Devices) or FLEXstation™ (Molecular Devices) using a fluorescent calcium-sensitive dye. Fluorescent calcium-sensitive dyes are available in a range of affinities to calcium ions (10⁻⁸ to 10⁻⁵ M), excitation and emission spectra, and chemical forms. They show different temporal resolution (from milliseconds, like Fluo-3, to tens of seconds, like Fura-2), and different degrees of accuracy for each range of calcium concentration. Dyes such as Fluo-3 are suitable for measuring small, fast transients associated with calcium ‘sparks’. These calcium dyes are usually used in conjunction with a FLIPR-type kinetic fluorescent reader to achieve high-throughput, low-noise detection of both absolute levels and changes of cytosolic calcium concentration. FLIPR typically utilises a 384-well microplate format with a throughput of up to 10 plates per hour depending on the assay format. Other suitable fluorescent calcium-sensitive dyes include Fluo-4, Calcium Green and Calcium 3 (Molecular Devices). Suitable fluorescent Ca²⁺ indicators excited with UV light include Fura-2, Indo-1, Bis-Fura-2, Quin-2, Quin-2 AM, Fura-4F, Fura-5F, Fura-6F, Fura-FF, Indo-5F, BTC, Mag-Fura-2, Mag-Fura-5 and Mag-Indo-1 and related derivatives and conjugates thereof (Invitrogen); suitable fluorescent Ca²⁺ indicators excited with visible light include Fluo-3, Fluo-4, Rhod-2, X-Rhod-1, Fluo-5F, Fluo-4FF, Fluo-5N, Mag-Fluo-4, Rhod-5N, Rhod-FF, X-Rhod-5F, X-Rhod-FF, Calcium Green-1, Calcium Green-2, Calcium Green-5N, Magnesium Green, Calcium Yellow, Calcium Orange, Calcium Crimson, Oregon Green 488 BAPTA-1, -2, -6F, and -5N, Fura Red and Calcein and related derivatives and conjugates thereof (Invitrogen).

In a less preferred embodiment, a non-fluorescent calcium-sensitive dye is used in step (b), and step (f) comprises measuring the absorbance spectrum characteristic of the dye-calcium complex. Suitable colorimetric assays are known in the art and are described, for example, in U.S. Pat. No. 5,262,330.

Typically, the method also comprises the step of measuring the fluorescence from the intracellular calcium-sensitive dye before exposure to the test compound and/or the calcium and potassium ions, thereby to obtain a baseline measurement.

It is preferred that the method further comprises the step of comparing the rate and/or amount of calcium ion exchange determined in the presence of the test compound in step (f) with a control measurement.

Thus, the method typically comprises the step of identifying whether the rate and/or amount of NCKX-mediated calcium ion exchange across the cell membrane has increased, decreased or stayed the same in response to exposure to the test compound in comparison to the control measurement.

It is appreciated that there are a number of control measurements that may be useful in the screening method of this inventions. For example, in one embodiment, the control measurement may be obtained by performing steps (a), (b), (c), (e) and (f) in the absence of a test compound, thereby to determine the rate and/or amount of calcium ion exchange across the cell membrane in the absence of the test compound.

An additional or alternative control measurement may be obtained by performing steps (b), (c), (d), (e) and (f) on a control cell, the cell membrane of which does not contain an NCKX polypeptide or a functionally equivalent variant thereof, thereby to determine the rate and/or amount of calcium ion exchange across the cell membrane in the absence of the NCKX polypeptide or the functionally equivalent variant.

A further additional or alternative control measurement may be obtained by performing steps (a), (b), (c), (e) and (f) in the presence of a positive control compound known to stimulate NCKX-mediated calcium ion exchange across a cell membrane, thereby to determine the rate and/or amount of calcium ion exchange across the cell membrane in the presence of the positive control compound. This may be useful, for example, when screening for a compound that has an equivalent or greater effect to that of the positive control compound.

A still further additional or alternative control measurement may be obtained by performing steps (a), (b), (c), (e) and (f) in the presence of a negative control compound known to inhibit NCKX-mediated calcium ion exchange across a cell membrane, thereby to determine the rate and/or amount of calcium ion exchange across the cell membrane in the presence of the negative control compound. This may be useful, for example, when screening for a compound that has an equivalent or greater effect to that of the negative control compound.

It is appreciated that the presence of selective blockers of other ion transporters, exchangers and channels may be useful to determine whether or not the test compound modulates Ca²⁺ exchange via the NCKX molecule. For example, Nifedipine (e.g. 10 μM) may be used to block voltage-gated Ca²⁺ channels, SKF-96365 (e.g. 10 μM) may be used to block receptor-operated Ca²⁺ channels, ouabain (e.g. 1 mM) may be used to block the Na⁺ pump and KB-R7943 (e.g. 1 μM) may be used to block NCX-type exchangers (Dong et al, 2006).

The method may also include the step of testing the selectivity of the test compound (i.e. whether or not it affects any other ion channels) by using broadly known techniques such as electrophysiological methods such as patch clamping.

Typically, the test compound is a peptide, for example a di- or tri-peptide, a nucleic acid molecule, a small organic molecule or a small inorganic molecule, including natural compounds, mixtures of natural compounds and extracts thereof, and combinations thereof.

In an embodiment, the initial screening is not conducted by testing single test compounds, but by testing larger groups of compounds simultaneously for the ability to modulate NCKX-mediated calcium ion exchange across a membrane. Thus, in this embodiment, step (d) comprises simultaneously exposing the cell to a plurality of test compounds. Since a positive result cannot then be ascribed to any of these compounds individually, the method typically further comprises the step of isolating and/or identifying individual test compounds within the plurality of test compounds.

In a further embodiment, the method further comprises the step of individually retesting the isolated and/or identified test compounds for the ability to modulate NCKX-mediated calcium ion exchange across a cell membrane.

Preferably, the method further comprises the step of identifying a test compound that is found to have the ability to modulate NCKX-mediated calcium ion exchange across the cell membrane.

In an advantageous embodiment, the identified compound is tested for its selectivity against one or more other calcium exchanger molecules, such as other NCKX polypeptides. Preferably, a compound which is selective for a desired NCKX polypeptide is thereby identified.

It is appreciated that this method may be a drug screening method, a term well known to those skilled in the art, and the test compound may be a drug-like compound or a lead compound for the development of a drug-like compound. The test compound may also be a cosmetically-useful compound or a lead compound for the development of a cosmetically-useful compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 Daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include the meaning that the compound, whilst not itself suitable for use as a drug or cosmetic compound (for example because it is only weakly potent against its intended target, non-selective in its action, unstable, poorly soluble, difficult to synthesise or has poor bioavailability), may provide a starting-point for the design of other compounds that may have more desirable characteristics.

For example, it may be desired to identify a compound that selectively modulates NCKX5-mediated ion exchange. However, for convenience, the initial screen may be carried out using an NCKX polypeptide other than NCKX5, such as NCKX2, and hence an NCKX2 modulator is identified as a lead compound for identifying an NCKX5 modulator. Alternatively, when the screening is conducted using NCKX5, an identified compound may be active at NCKX5 in addition to other NCKX polypeptide(s). Again, the identified compound may be a lead compound for the identification of an NCKX5-specific modulator of calcium ion exchange across a cell membrane.

Thus, in an embodiment, the identified compound is modified, and the modified compound is tested for the ability to modulate NCKX-mediated calcium ion exchange across a cell membrane. In particular, the modified compound may be tested for the ability to modulate calcium ion exchange, mediated by a desired

NCKX polypeptide, across a cell membrane. Advantageously, the modified compound may be tested for its selectivity against one or more other calcium exchanger molecules, such as NCX or CCX molecules (Lytton, 2007) or other NCKX polypeptides. Preferably, a compound which is selective for a desired NCKX polypeptide is thereby identified.

The desired NCKX polypeptide may be NCKX1, NCKX2, NCKX3, NCKX4 or NCKX5.

In a preferred embodiment, the desired NCKX polypeptide is NCKX5.

In an embodiment, the method of the first aspect of the invention can be used to identify a compound that modulates pigmentation, i.e. that increases or decreases pigmentation.

It is appreciated that NCKX5 is involved in skin, hair and eye pigmentation (Lamason et al (2005); Example 11). Thus, by modulating pigmentation we include modulating skin pigmentation, hair pigmentation and eye pigmentation. Accordingly, the method of the first aspect of the invention can be used to identify a test compound that increases or decreases skin pigmentation or that alters the melanin composition of skin, or to identify a test compound that increases or decreases hair pigmentation or that alters the melanin composition of hair, or to identify a test compound that increases or decreases eye pigmentation or that alters the melanin composition of the eye/retina.

It is appreciated that when it is desired to identify a compound that modulates pigmentation, step (a) may comprise providing an adherent cell, the cell membrane of which comprises an NCKX5 polypeptide or a functionally equivalent variant of an NCKX5 polypeptide. However, as discussed above, the adherent cell in step (a) may comprise an NCKX polypeptide other than NCKX5 in the cell membrane. A compound thus identified may be a lead compound for the identification of an NCKX5 modulator, whether specific for the other NCKX polypeptide, or active at NCKX5 in addition to other NCKX polypeptide. Thus, typically, the identified compound would be modified and retested for the ability to modulate or to selectively modulate NCKX5-mediated calcium ion exchange across a cell membrane as discussed above.

Preferably, an increase in the rate and/or amount of NCKX5-mediated calcium ion (Ca²⁺) exchange across the cell membrane indicates the test compound may increase pigmentation, and a decrease in the rate and/or amount of NCKX5-mediated Ca²⁺ exchange across the cell membrane indicates the test compound may decrease pigmentation.

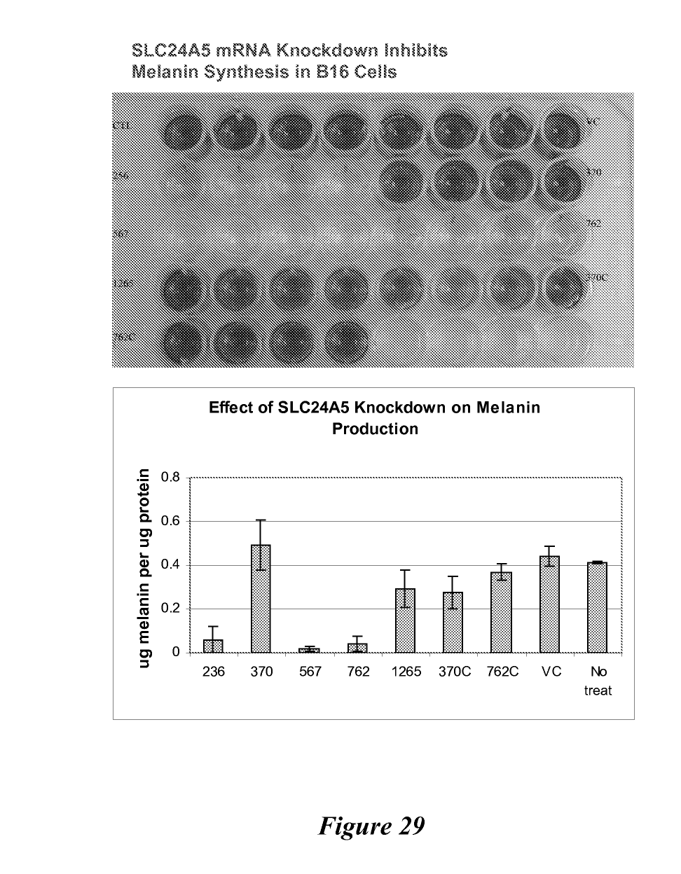

Preferably, the identified compound or the modified compound is further tested for the ability to modulate pigmentation. This can be done using a melanogenesis assay, for example using murine B16 cells which secrete melanin upon reaching confluence as described by Laskin et al (1982) J Cell Physiol 113: 481-486, and shown in FIG. 29. Briefly, the assay comprises incubating a test compound with a given number of cells for 2 days and assessing whether melanin secretion has been modulated (inhibited or stimulated) compared to a control. Alternatively, human melanocytes could be incubated with a test compound, melanin extracted and absorbance measured at 450 nm.

Uses of the compounds identified and isolated by the methods of the invention may be as inhibitors or activators of NCKX5 function.

It is appreciated that a compound identified in the screening methods of the first aspect of the invention which increases the rate and/or amount of NCKX5-mediated calcium ion exchange may be therapeutically useful for the treatment or prevention of a disease or condition characterised by reduced skin pigmentation, for the prevention of sun-induced skin damage, skin cancer and/or diseases characterised by vitamin D deficiency (e.g. osteoporosis). Such compounds may also benefit individuals with a disease or condition characterised by sensitivity to UV and/or visible light, for example porphyria and xeroderma pigmentosum. Thus the method may further comprise the step of testing an identified or modified compound which increases NCKX5-mediated calcium ion exchange and/or which increases pigmentation in a cellular, tissue or animal model of these diseases or conditions, as is known in the art.

A compound which increases the rate and/or amount of NCKX5-mediated calcium ion exchange may also be useful in a cosmetic formulation for increasing pigmentation, including skin, hair or eye pigmentation. Compounds that activate NCKX5 can be used to enhance pigment production in these tissues. For example, consumers with lighter skin may obtain a natural tan without the risks that arise from sun exposure (such as burning and the discomfort associated with that, skin ageing and skin cancer). The activators might also use a tanning product to prepare the skin for subsequent sun exposure, a so-called pre-sun treatment. Use of such a tanning product may reduce the effects over time of photoageing, and therefore indirectly will have a skin ageing benefit. For example, wrinkles, sallowness, sagging, fine lines, age spots or mottled pigmentation could be reduced. Individuals who are especially sensitive to UV and/or visible light, may use a tanning product to better protect themselves from sun exposure when they go outside. Thus the method may further comprise the step of testing an identified or modified compound which increases NCKX5-mediated calcium ion exchange and/or which increases pigmentation as a tanning agent, or as a hair or eye darkening agent, using methods well known in the art. The protection of such activators may extend to individuals for whom sun exposure is contra-indicated e.g. those who are especially sensitive to skin cancer, for example because of defective DNA repair mechanisms or phototoxic reactions.

It is appreciated that a compound which decreases the rate and/or amount of NCKX5-mediated calcium ion exchange across the cell membrane may be therapeutically useful for the treatment or prevention of a disease or condition characterised by elevated or excessive pigmentation. Diseases or conditions characterised by elevated or excessive pigmentation include melanoma, melasma, age spots, and increased pigmentation through inflammation. Thus the method may further comprise the step of testing an identified or modified compound which decreases NCKX5-mediated calcium ion exchange and/or which decreases pigmentation in a cellular, tissue or animal model of these diseases or conditions, as is known in the art.

A compound which decreases the rate and/or amount of NCKX5-mediated calcium ion exchange may be useful in a cosmetic formulation for reducing pigmentation, including skin, hair or eye pigmentation. Compounds that inhibit NCKX5 can be used to decrease pigment production in these tissues. For example, in darker skin, inhibition of the exchanger will reduce pigment production and lighten the skin, which may be desirable in certain societies. Such inhibitors may also be used for treating age spots. NCKX5 inhibitors may also enhance UV dependent vitamin D synthesis in skin as a reduction in melanin will reduce melanin-induced blockage of the UV dependent synthesis of vitamin D in skin which has general health benefits including on bone, e.g. in improving and/or preventing osteoporosis benefits over time. Thus the method may further comprise the step of testing an identified or modified compound which decreases NCKX5-mediated calcium ion exchange and/or which decreases pigmentation as a lightening agent, using methods well known in the art.

Inhibitors and activators of NCKX5 might also be used to change the pigmentation or composition of skin, hair or eye melanin so that the skin, hair or eye colour is altered. Inhibitors and activators of NCKX5 might also be used to change the pigmentation in animals so that the coat or skin colour of the animal is lightened or darkened. The uses of pigmentation changes in animals may range from protection of animals against sunburn to altering skin pigmentation for textiles such as leather. The term “altering the composition” denotes altering the colour and or melanin composition of skin by using inhibitors and activators of NCKX5. The melanin composition of the skin is determined by the proportions of eumelanin and pheomelanin that is present in the tissue. Thus the method may further comprise the step of testing an identified or modified compound which modulates NCKX5-mediated calcium ion exchange and/or which modulates pigmentation for the ability to affect coat or skin colour in animals.

Since NCKX5 is expressed in the retinal pigmented epithelium, NCKX1 is expressed in retinal rods, and NCKX2 is expressed in retinal cones and ganglia, modulators of NCKX-mediated ion exchange may also be useful for treating retinal disease. Thus, a compound identified in the screening methods of the first aspect of the invention may also be useful in the treatment or prevention of a retinal disease such as age-related macular degeneration. Thus the method may further comprise the step of testing an identified or modified compound which modulates NCKX1-, NCKX2- or NCKX5-mediated calcium ion exchange in a cellular, tissue or animal model of these diseases or conditions, as is known in the art.

Study of the NCKX2 knock-out mouse suggests a role for NCKX2 in memory and learning (Kiedrowski et al (2006) J Biol Chem 281: 6273-82). Thus, a compound identified in the screening methods of the first aspect of the invention may also be useful in stimulating or improving memory, or in the treatment or prevention of a neurodegenerative disease such as Alzheimer's disease (AD), Parkinson's disease (PD) and Huntington's disease (HD). Thus the method may further comprise the step of testing an identified or modified compound which modulates NCKX2-mediated calcium ion exchange in a cellular, tissue or animal model of these diseases or conditions, as is known in the art.

Since NCKX3 and NCKX4 are expressed and active in vascular smooth muscle cells, modulators of NCKX-mediated ion exchange may also be useful for treating disorders of vascular smooth muscle cells. Thus, a compound identified in the screening methods of the first aspect of the invention may also be useful in the treatment or prevention of a disorder of vascular smooth muscle cells such as hypertension, atherosclerosis, restenosis, diabetes, renal pathologies, asthma, obstructive bladder disease, and various gastrointestinal and reproductive disorders, and solid tumours (Owens et al (2004) Physiol Rev 84: 767-801). Thus the method may further comprise the step of testing an identified or modified compound which modulates NCKX3- or NCKX4-mediated calcium ion exchange in a cellular, tissue or animal model of these diseases or conditions, as is known in the art.

In a further advantageous embodiment, the identified compound or the modified compound is tested for toxicity in a cellular, tissue or animal model of toxicity. Many suitable cell proliferation/toxicity assays are known in the art and include the WST-1 assay (Roche) or a colorimetric MTT assay which relies on the reduction of a tetrazolium component MTT by the mitochondria of viable cells.

Optionally, the method further comprises the step of synthesising and/or purifying the identified compound or the modified compound. By a “purified” compound we mean that the compound comprises at least 95% by weight, more preferably at least 99% by weight of the molecules present (not including water, buffers, and other small molecules when the compound is present in solution). Preferably, the purified compound is pure to a recognised pharmaceutically or cosmetically acceptable standard.

Further optionally, the method still further comprises the step of formulating the identified compound or the modified compound into a pharmaceutical formulation.

Further optionally, the method still further comprises the step of formulating the identified compound or the modified compound into a cosmetic formulation.

In a further aspect of the invention there is provided a compound identified by the method of the first aspect of the invention.

All of the documents referred to herein are incorporated herein, in their entirety, by reference.

The listing or discussion of an apparently prior-published document in this specification should not necessarily be taken as an acknowledgement that the document is part of the state of the art or is common general knowledge

Examples embodying certain preferred aspects of the invention will now be described with reference to the following Figures in which:

FIG. 1—NCKX5 (SLC24A5) transcript reference sequence. Nucleotide and amino acid sequence of NCKX5 showing the position of the Ala/Thr SNP linked to skin pigmentation.

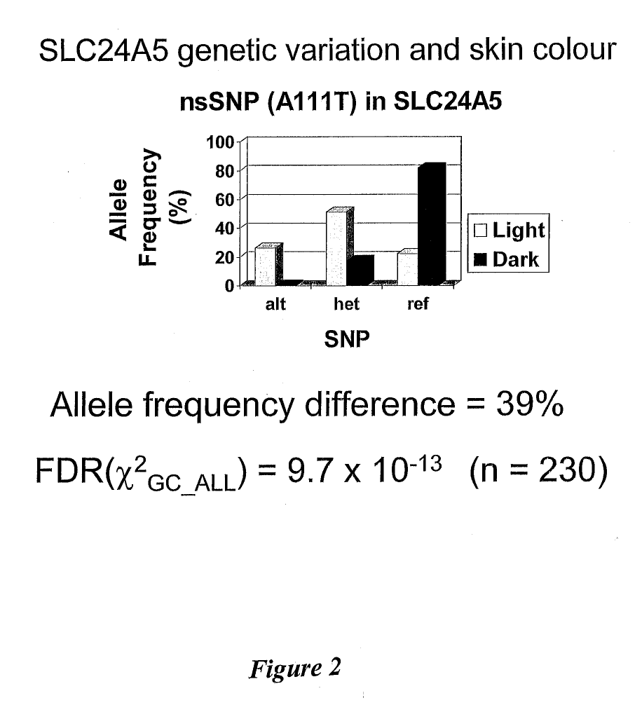

FIG. 2—Distribution of alleles in relation to human skin pigmentation. Graph showing the frequency of alleles and relationship skin colour. The reference allele is the Thr containing and the alternate copy is Ala containing. Hence, Ala allele is shown to be predominantly found in darker skins

FIG. 3—NCKX5 homology and position of Ala/Thr variation

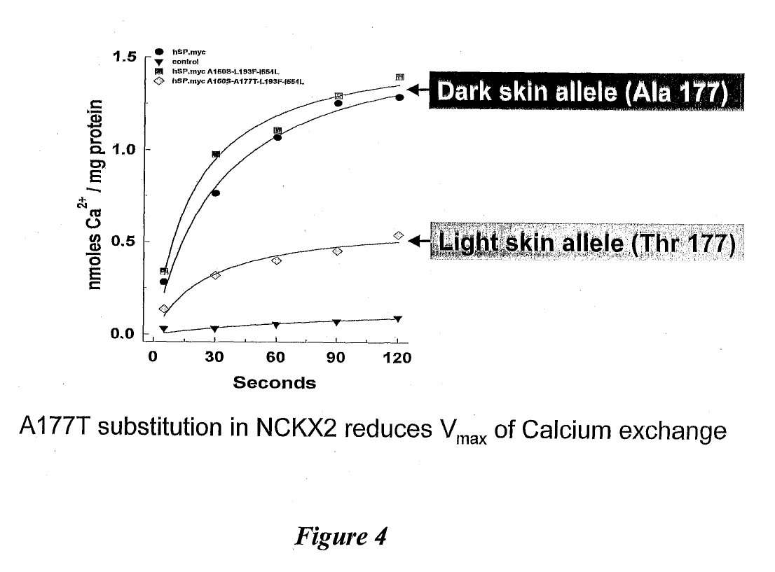

FIG. 4—Calcium extrusion using modified NCKX2. The Figure shows the amount of calcium extrusion due to the two variants (Ala and Thr) of an NCKX2 molecule that has been modified to resemble NCKX5. A1a177 (equivalent to Ala111 in NCKX5) in NCKX2 (dark skin allele) is associated with increased calcium exchange.

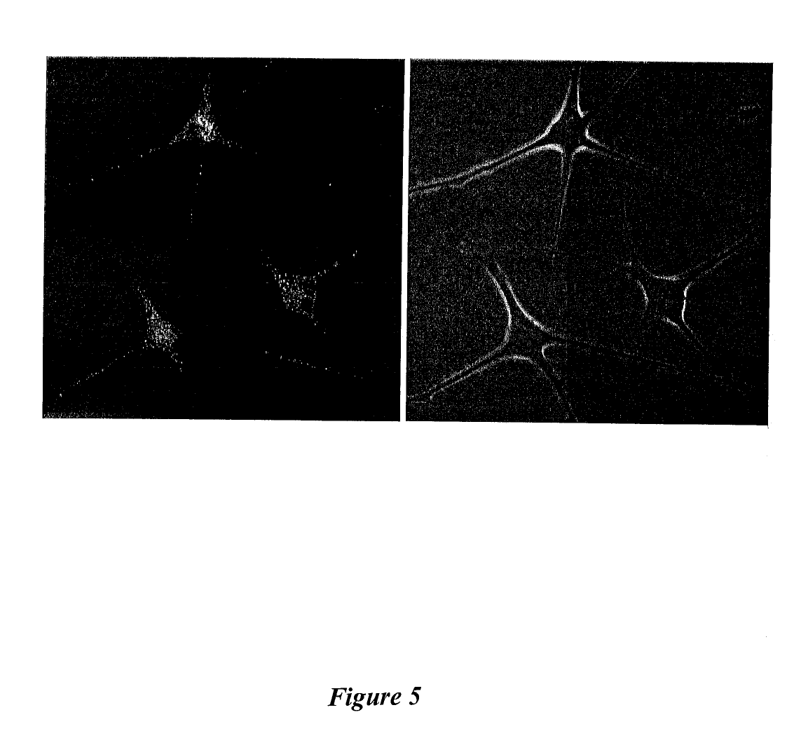

FIG. 5—Staining of melanocytes. Melanocytes stained with NCKX5 antisera show punctuate cytoplasmic staining and peri-nuclear staining

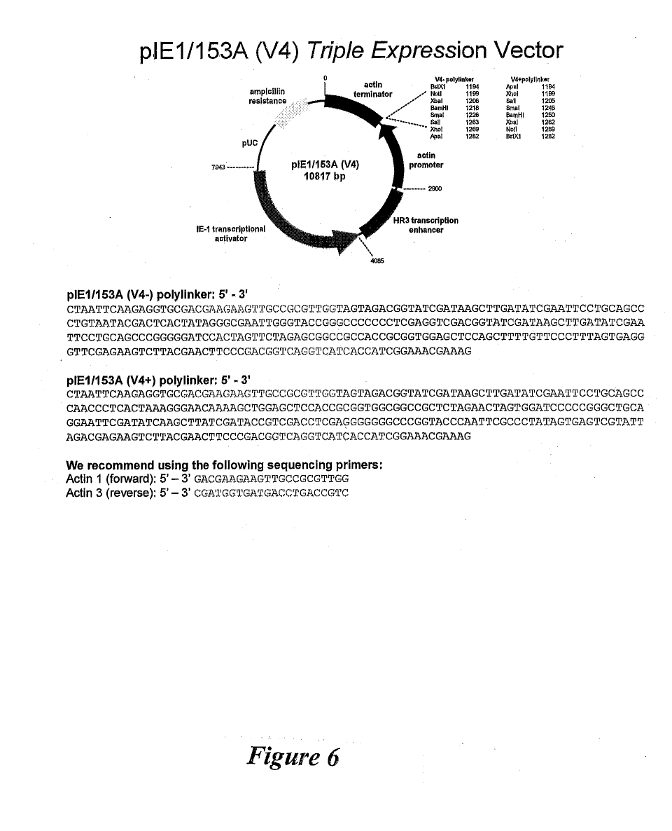

FIG. 6—pIE1/1534 vector map

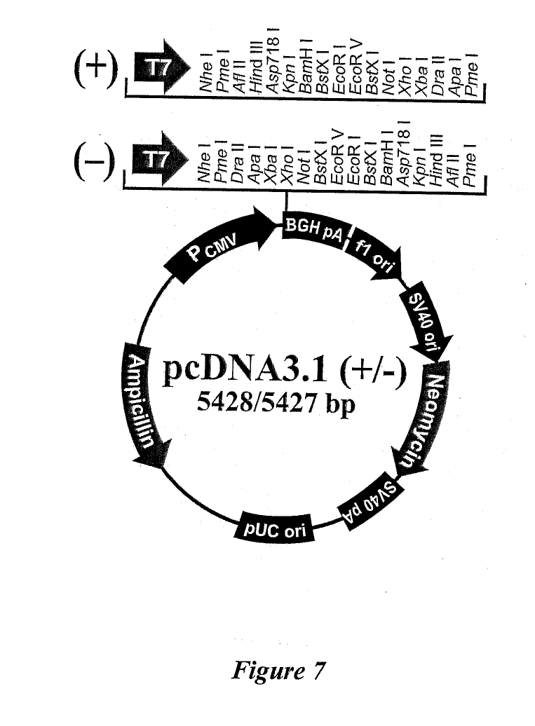

FIG. 7—pcDNA3.1 vector map

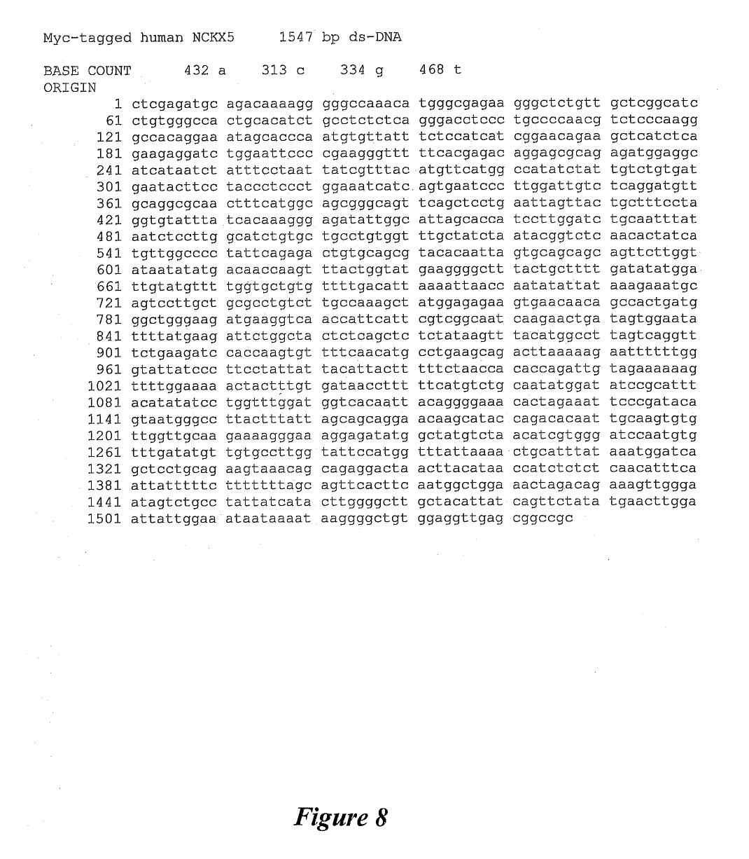

FIG. 8—Myc tagged human NCKX5 nucleotide sequence

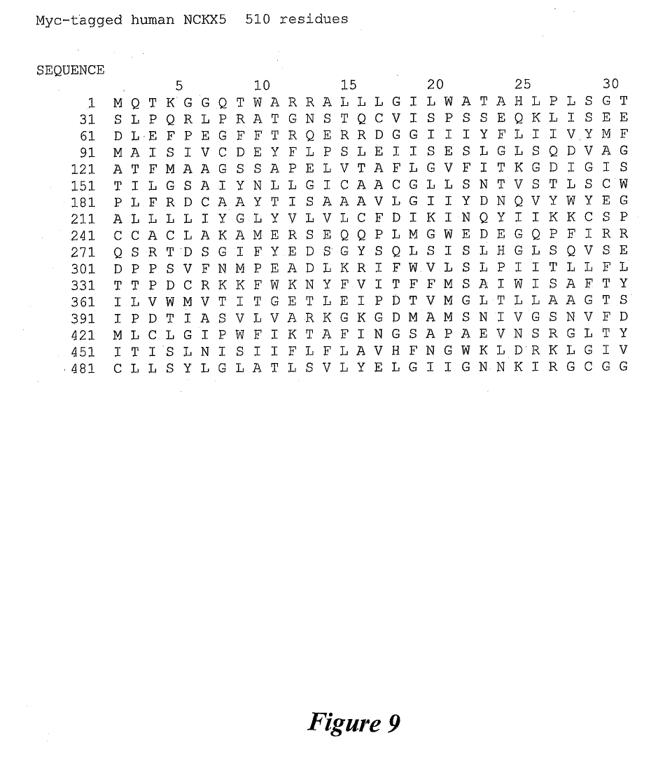

FIG. 9—Myc tagged human NCKX5 amino acid sequence

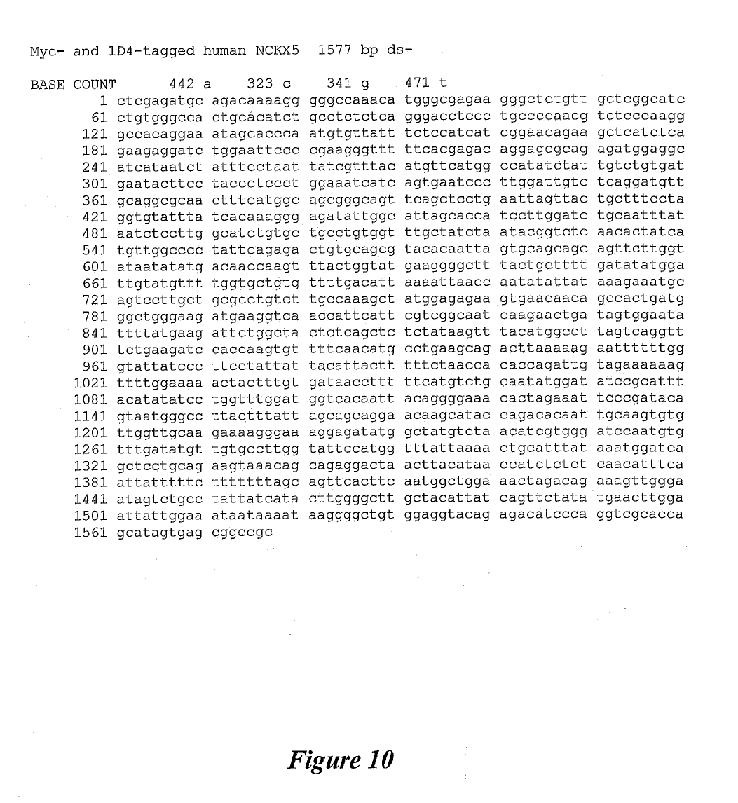

FIG. 10—Myc and 1D4 tagged human NCKX5 nucleotide sequence

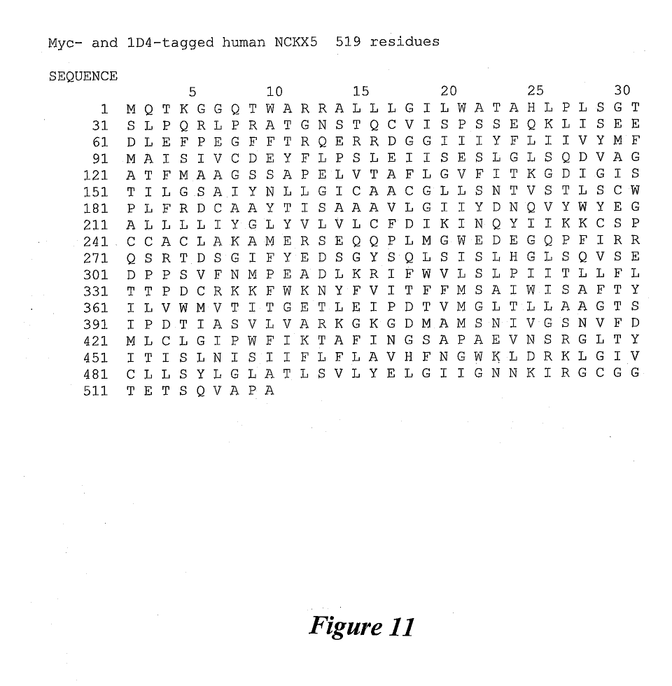

FIG. 11—Myc and 1D4 tagged human NCKX5 amino acid sequence

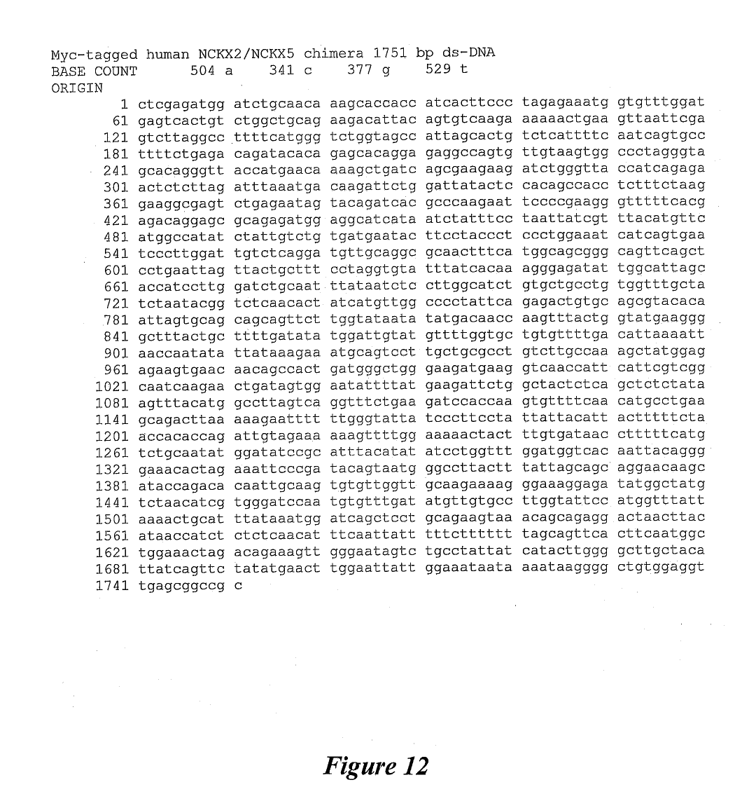

FIG. 12—Myc tagged human NCKX2/NCKX5 chimera nucleotide sequence

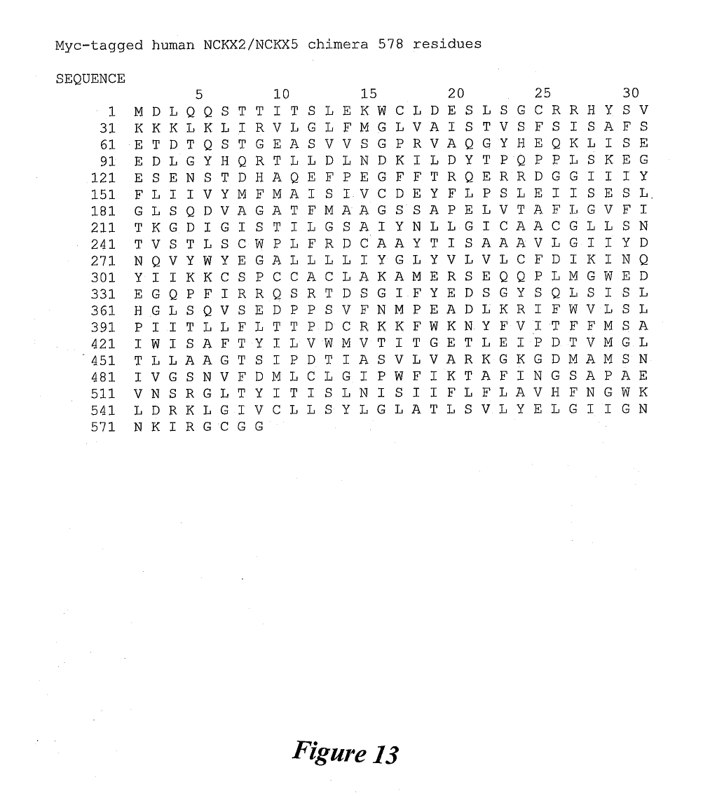

FIG. 13—Myc tagged human NCKX2/NCKX5 chimera amino acid sequence

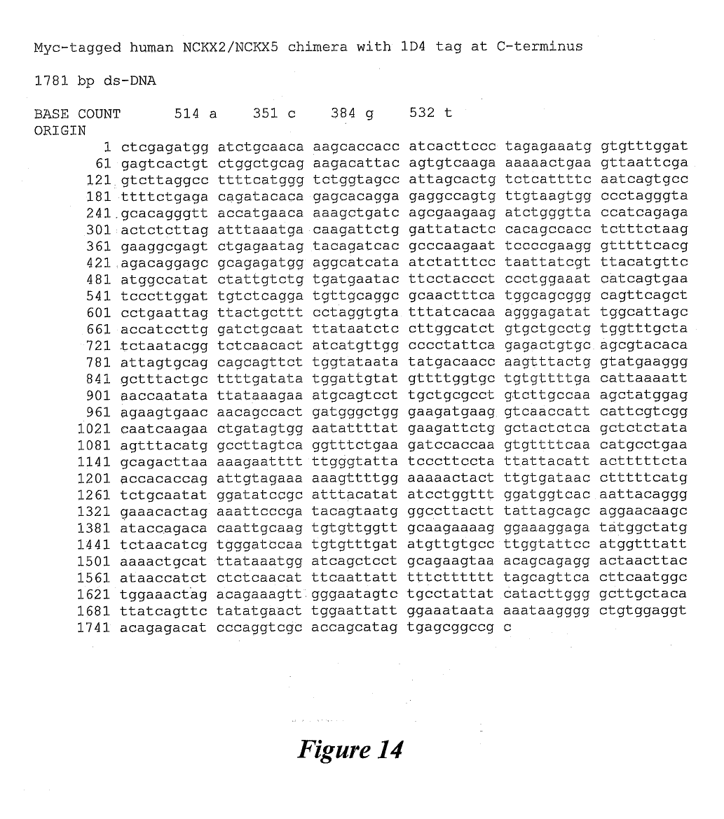

FIG. 14—Myc tagged human NCKX2/NCKX5 chimera nucleic acid molecule with ID4 tag at C-terminus

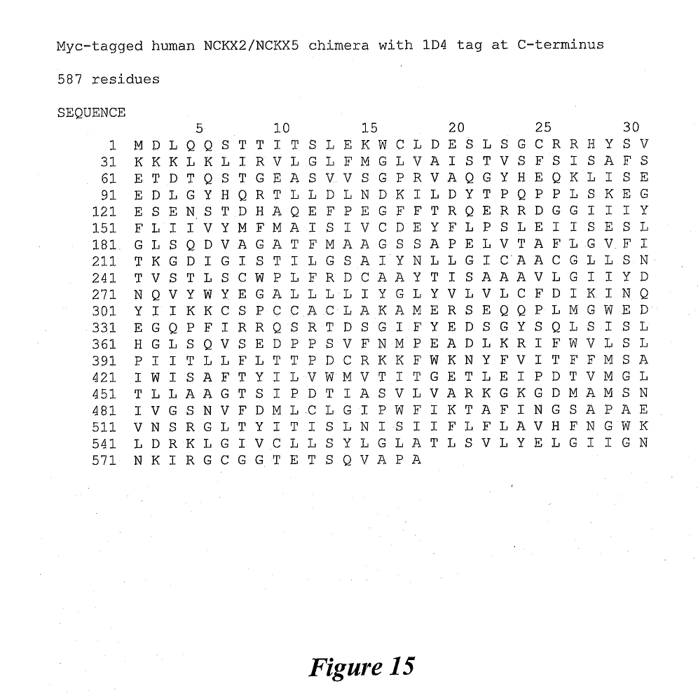

FIG. 15—Myc tagged human NCKX2/NCKX5 chimera amino acid sequence with ID4 tag at C-terminus

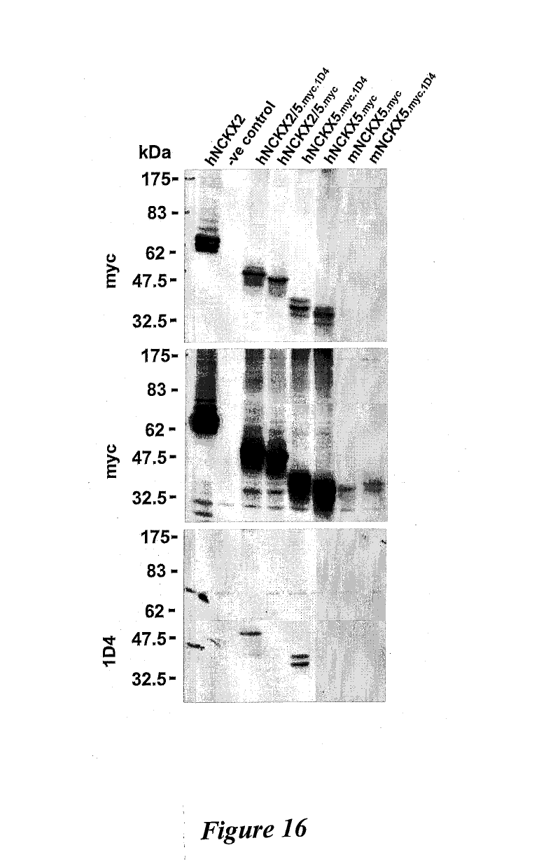

FIG. 16—Western blot of NCKX5 expression (Includes NCKX5 and NCKX5/NCKX2 chimeras and 1D4 and myc tagged versions)

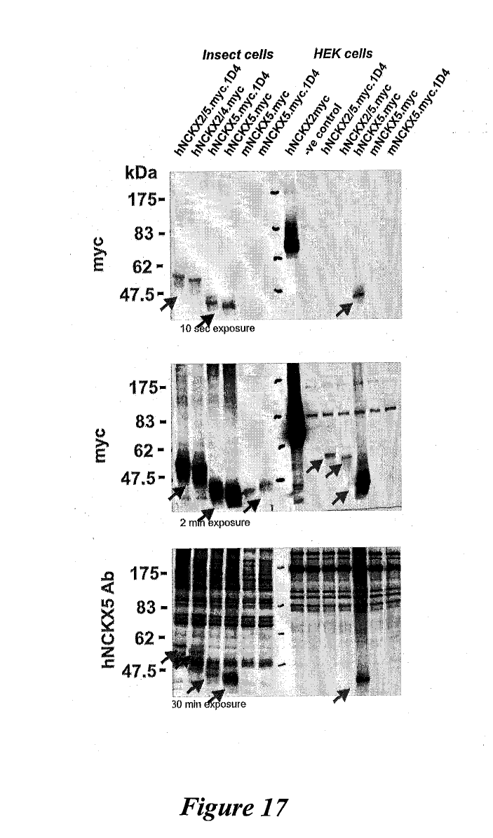

FIG. 17—Western blot of NCKX5 expression (Includes NCKX5 and NCKX5/NCKX2 chimeras and 1D4 and myc tagged versions) in High Five™ insect cells and HEK cells.

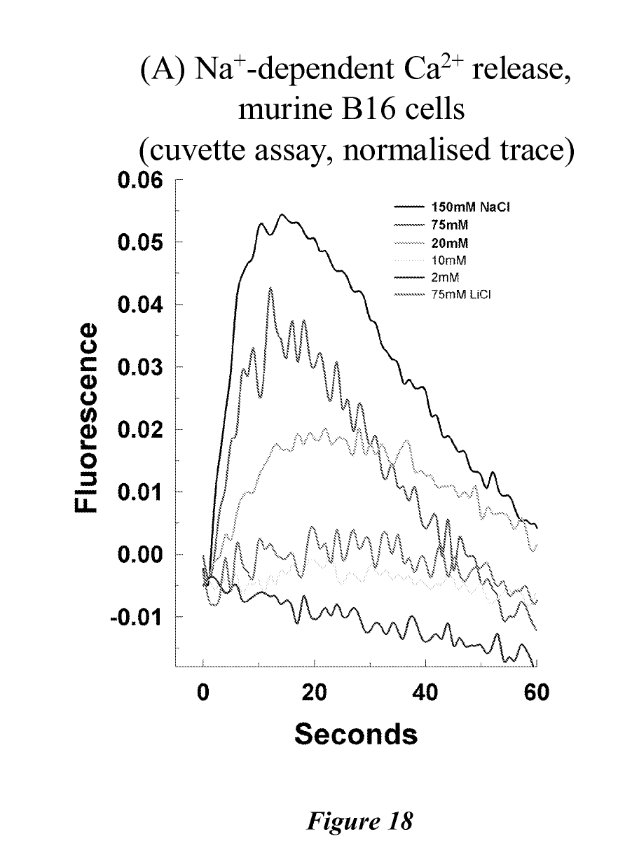

FIG. 18—Na⁺ dose-dependently induces intracellular Ca²⁺ release in B16 cells. Detected by cuvette method.

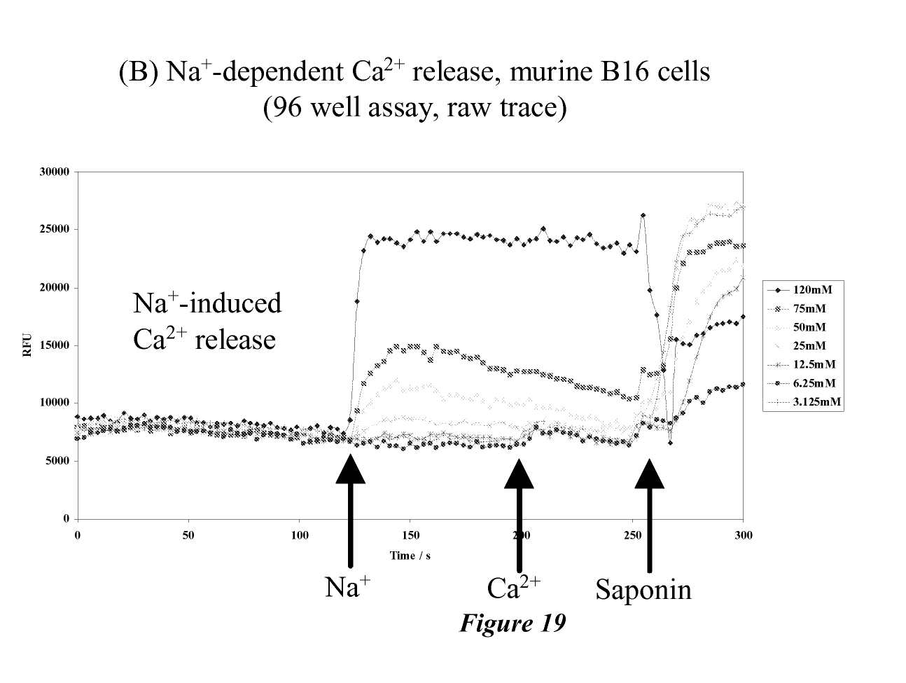

FIG. 19—Na⁺ dose-dependently induces intracellular Ca²⁺ release in B16 cells. Detected by 96 well assay

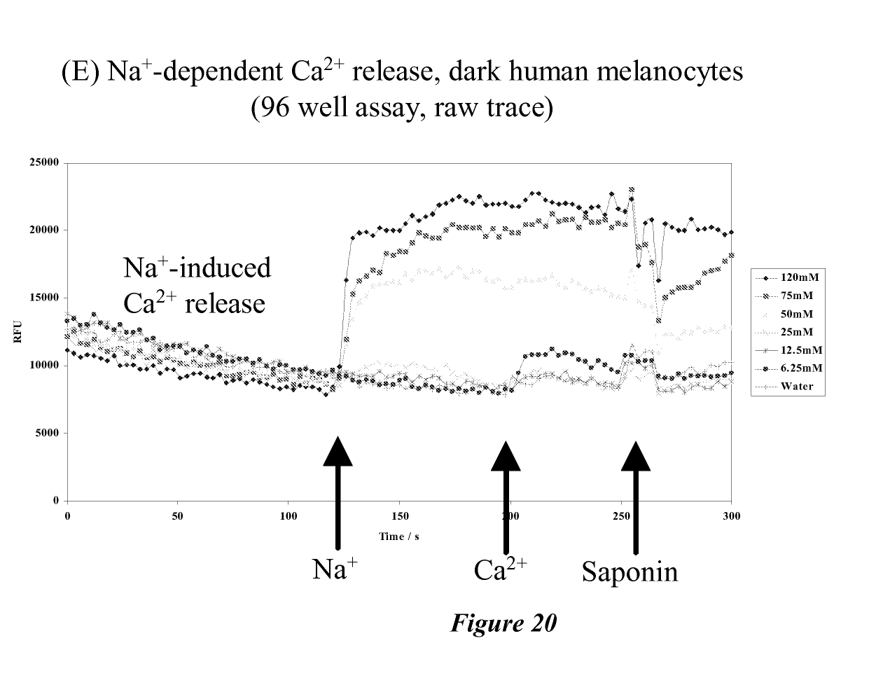

FIG. 20—Na⁺ dose-dependently induces intracellular Ca²⁺ release in B16 cells. Dose-dependent Na⁺-induced release of intracellular Ca²⁺ in dark human melanocytes by 96 well method.

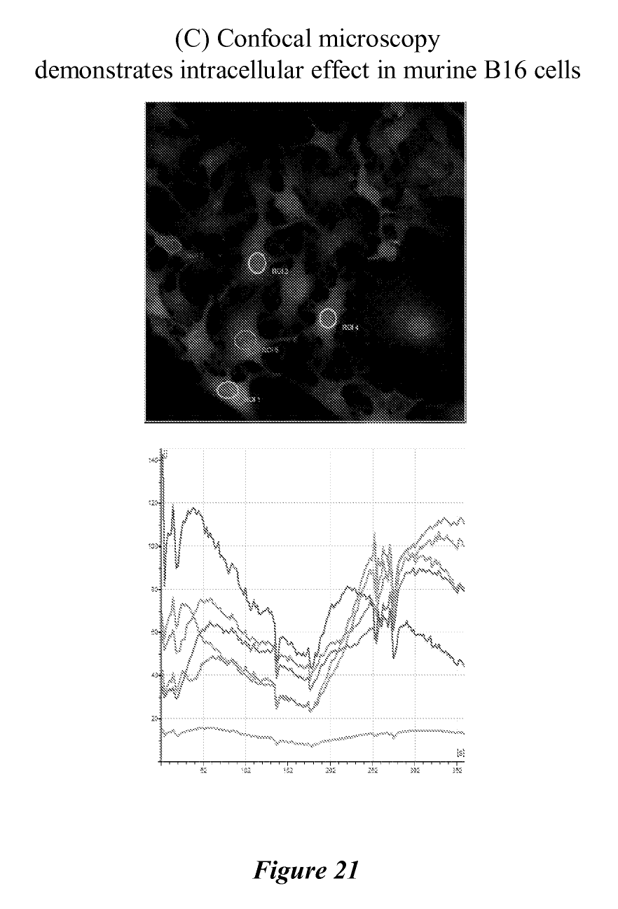

FIG. 21—Na⁺ dose-dependently induces intracellular Ca²⁺ release in B16 cells. Detected by confocal microscopy.

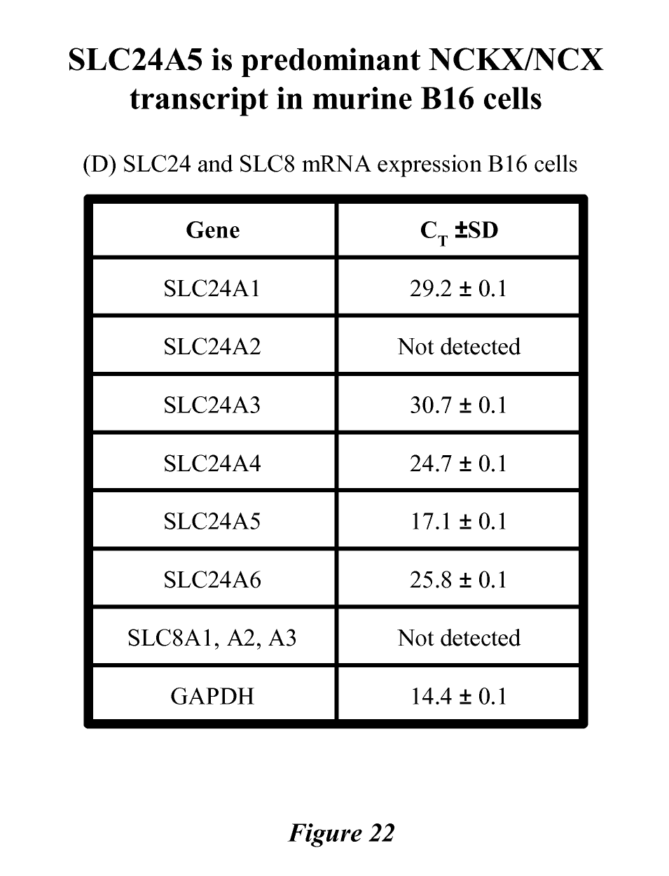

FIG. 22—Na⁺ dose-dependently induces intracellular Ca²⁺ release in B16 cells. mRNA expression of SLC24 and SLC8 in B16 cells as detected by real-time PCR following reverse-transcription of 1 μg RNA.

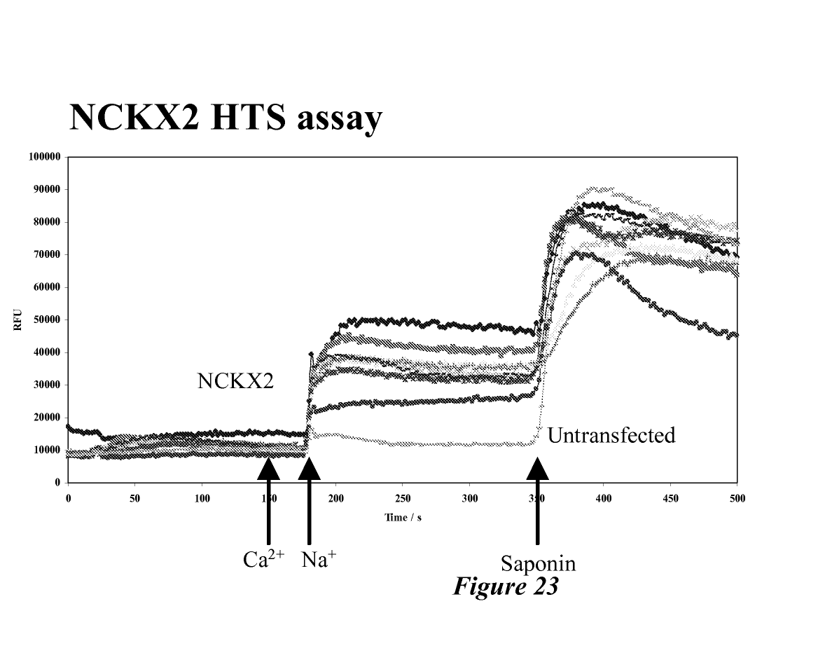

FIG. 23—A heterologous NCKX2 assay. A heterologous NCKX2 assay scaled to high throughput screening format being tested in the Amaxa 96 well Nucleofection device.

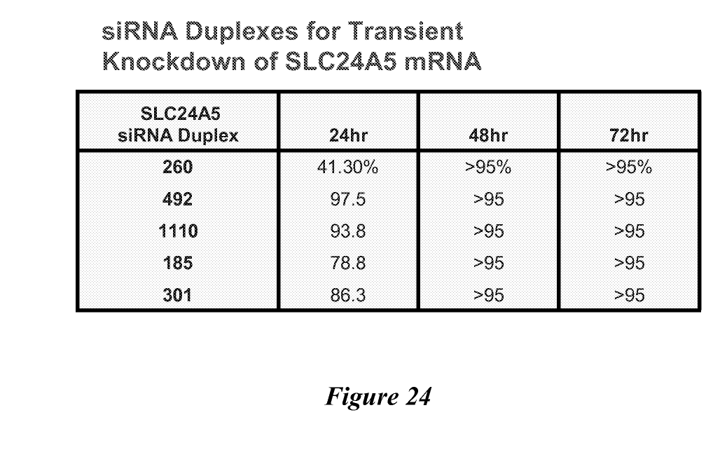

FIG. 24—Table showing decrease in SLC24A5 levels. 5 separate siRNA duplexes (designed by Invitrogen) decrease SLC24A5 mRNA levels by 95% or more in human primary melanocytes. This effect can be maintained for up to 10 days with re-transfection (data not shown).

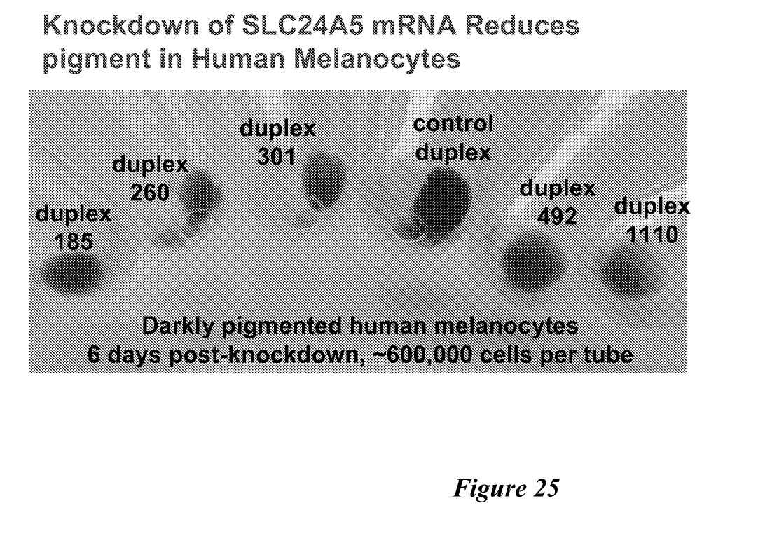

FIG. 25—Knockdown of SLC24A5 mRNA reduces pigment in human melanocytes. SLC24A5 knockdown followed by normalisation of cell numbers (by coulter counting) and centrifugation to pellet the melanocytes provides qualitative evidence that SLC24A5 is involved in a melanogenic process.

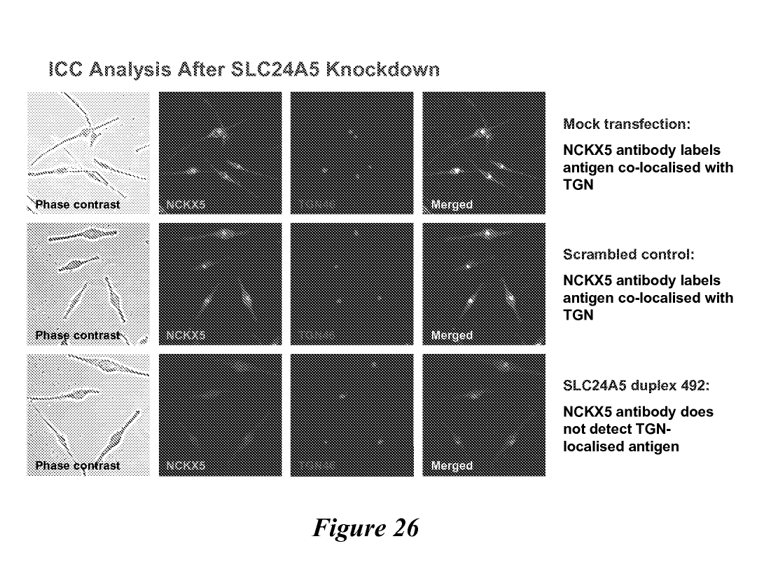

FIG. 26.—ICC Analysis after SLC24A5 knockdown. A 5 day knock down (using duplex 492 and controls) was conducted using darkly pigmented primary human melanocytes (Cascade). Primary antibodies for detection: Rabbit anti-NCKX5 cytosolic loop (raised using the peptide sequence DEGQPFIRRQSRTDSG) and sheep pAb anti-TGN46; Labelling was via Alexa Fluor® 488 anti-rabbit and 633 anti-sheep IgG's. The anti-NCKX5 polyclonal antibody localises within the TGN.

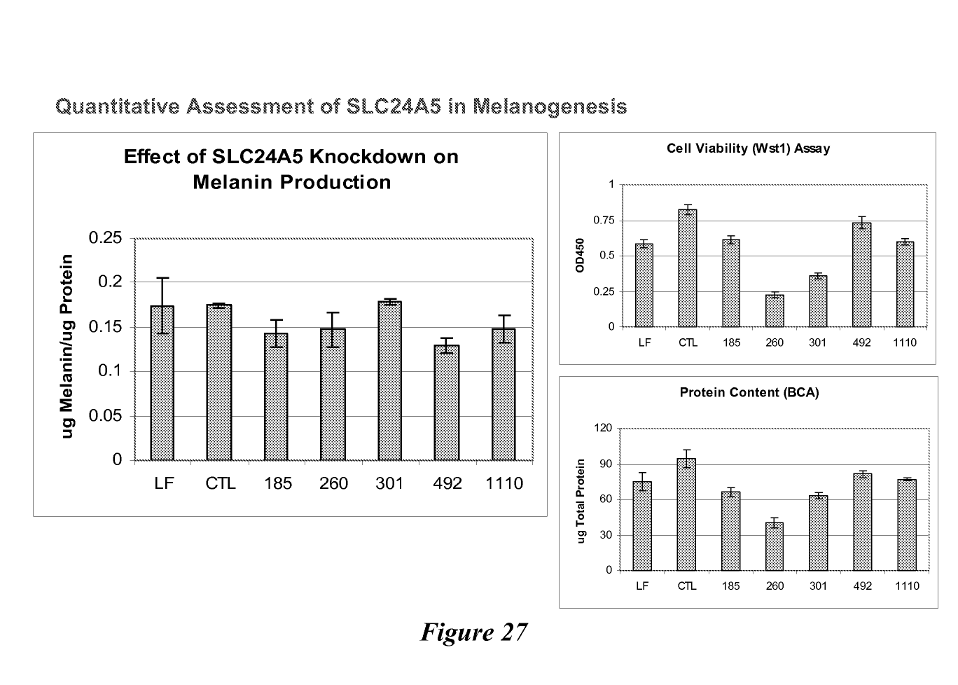

FIG. 27—Quantitative assessment of melanogenesis. A decrease in post-treatment melanin content is noted for duplex (492) in lightly pigmented human melanocytes (replicated ×3).

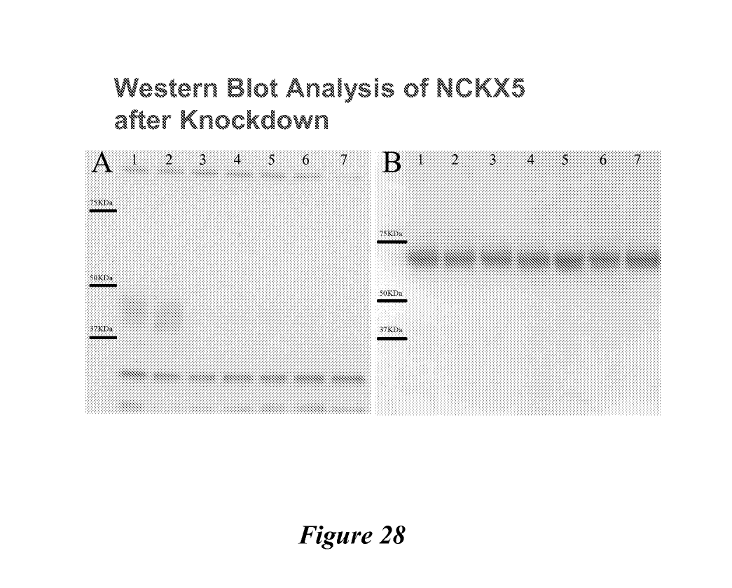

FIG. 28—Western blot analysis of NCK5 after knockdown. Using Western blotting we have investigated NCKX5 protein expression 5 days after siRNA mediated knockdown (all 5 siRNA duplexes and controls were tested). Western blot was also used to evaluate the expression and processing (maturation) of tyrosinase (tyr) in the same samples. SDS-PAGE (10% PA for NCKX5, 4-12% for tyr, 5 μg protein/lane). A) Western blot for NCKX5: Detection via rabbit anti-NCKX5 pAb (anti C-terminus peptide). B) Western blot for tyr, via goat pAb (Santa Cruz). 1=No treatment, 2=scrambled siRNA control, 3-7=siRNA's 185, 260, 301, 492, 1110. Western blot A) shows 2 bands, of approx. 42 and 44 kDa (significantly smaller than the predicted MW for NCKX5) in control wells. These bands are absent in the samples where SLC24A5-specific duplexes were used (lanes 3-7). It is possible that the doublet represents the presence of alternate SLC24A5 splice variants. The expression and processing of tyrosinase after knockdown (B) suggests that NCKX5 does not grossly alter the expression and/or maturation of tyrosinase, the rate limiting enzyme for melanin biosynthesis.

FIG. 29—SLC24A5 mRNA knockdown inhibits melanin synthesis in B16 cells. Cells were transfected (lipofectamine 2000) with siRNA duplexes or controls for 8 hours. Cells were cultured for a further 3 days prior to analyses.

Media was removed and melanin quantified by OD450. Cell viability measured using Wst1 proliferation reagent (Roche) before lysis using 1% triton ×100. Protein content of each well was determined by BCA assay. The experiment (in quadruplicate) was reproduced three times, consecutively. 3 siRNA duplexes (256, 567 and 762) visibly decreased melanin synthesis.

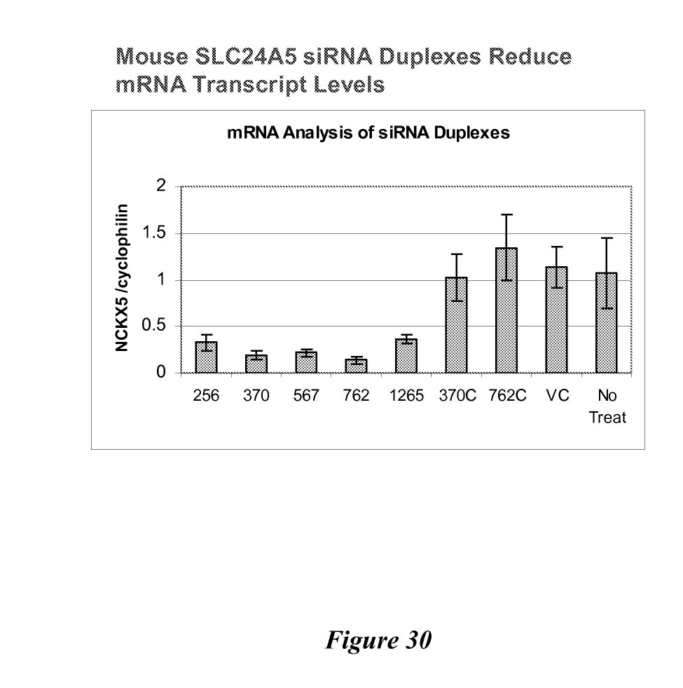

FIG. 30—Mouse SLC24A5 siRNA Duplexes Reduce mRNA Transcript Levels. All 5 siRNA duplexes were shown to be capable of achieving SLC24A5 mRNA knockdown in mouse B16 melanocytes. 3 duplexes were shown to be capable of reducing mRNA levels by more than 80% under the test conditions. Cells were transfected (Lipofectamine 2000) with 50 nM siRNA duplex or control for 8 hours. Cells were cultured for a further 24 hours prior to harvesting and real-time PCR analyses.

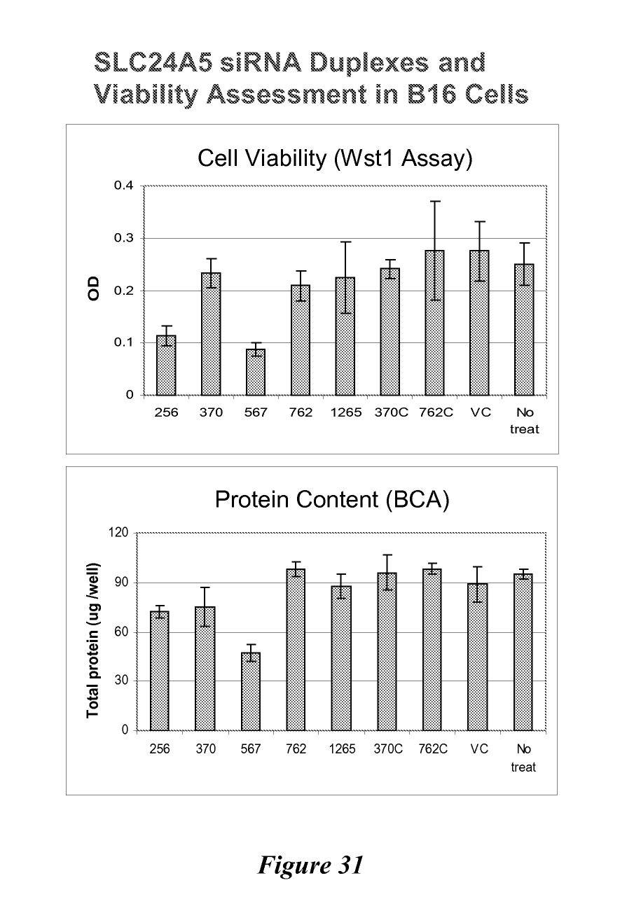

FIG. 31—SLC24A5 siRNA Duplexes and Viability Assessment in B16 Cells. Cell viability and protein content determination post-treatment could help to identify the manner in which SLC24A5 modulates pigment production in B16 melanocytes. Cell viability assessment at the end of each experiment suggested significant toxicity associated with the use of 2 duplexes (256 and 567). This conclusion is supported by data obtained for protein content. Duplex 762 appeared to reduce pigment production without affecting viability under the experimental conditions tested.

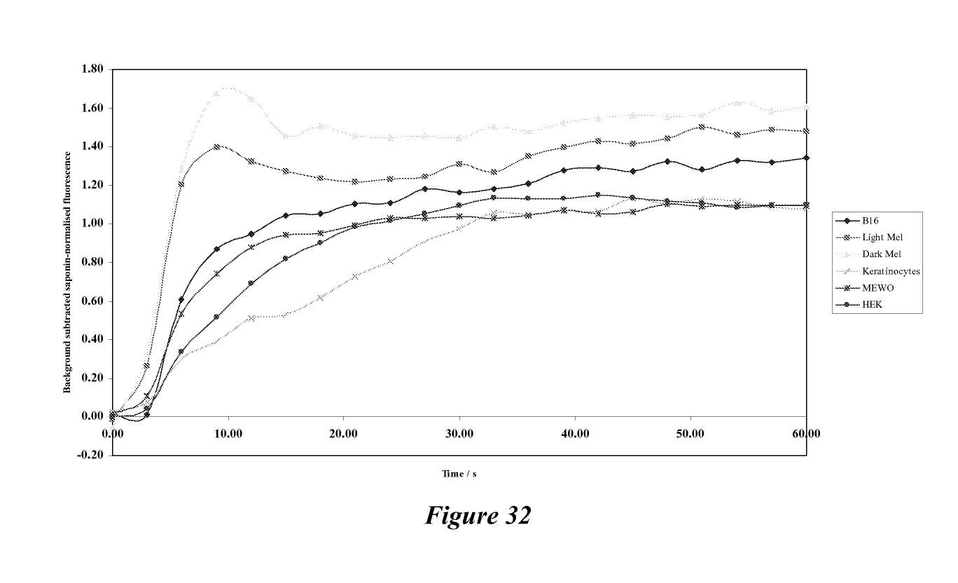

FIG. 32—Sodium-induced intracellular calcium release in various cells. 100,000 cells were plated overnight into a 96 well plate in triplicate. Intracellular calcium release in response to sodium was measured as described in methods. Data are background subtracted and saponin-normalised for comparison. Data are mean of 3 replicates.

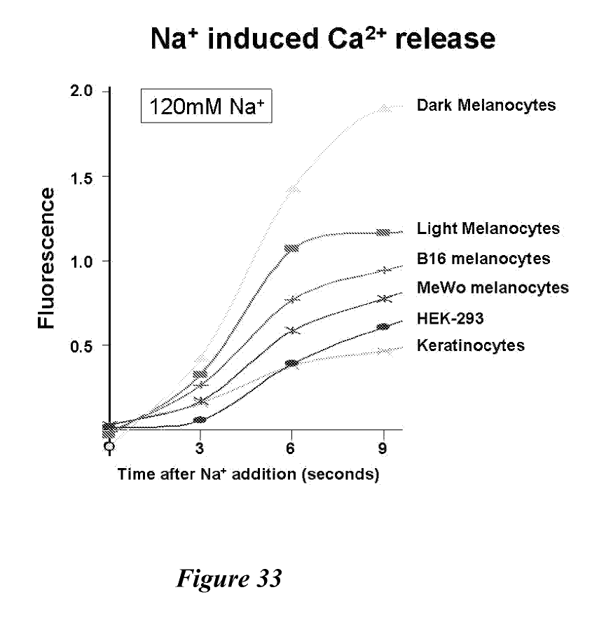

FIG. 33—Sodium-induced intracellular calcium release in various cells. 100,000 cells were plated overnight into a 96 well plate in triplicate. Intracellular calcium release in response to sodium was measured as described in methods. Data are background subtracted and saponin-normalised for comparison. Data are mean of 3 replicates.

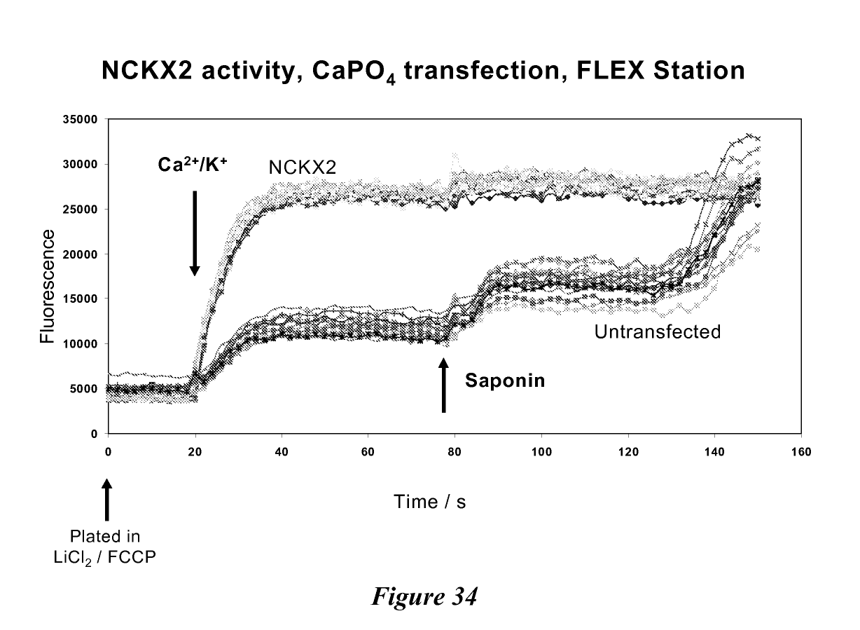

FIG. 34—NCKX2 assay. A high throughput NCKX2 assay of calcium chloride transfected and untransfected HEK 293 cells. Each trace on the graphs represents the signal from one well.

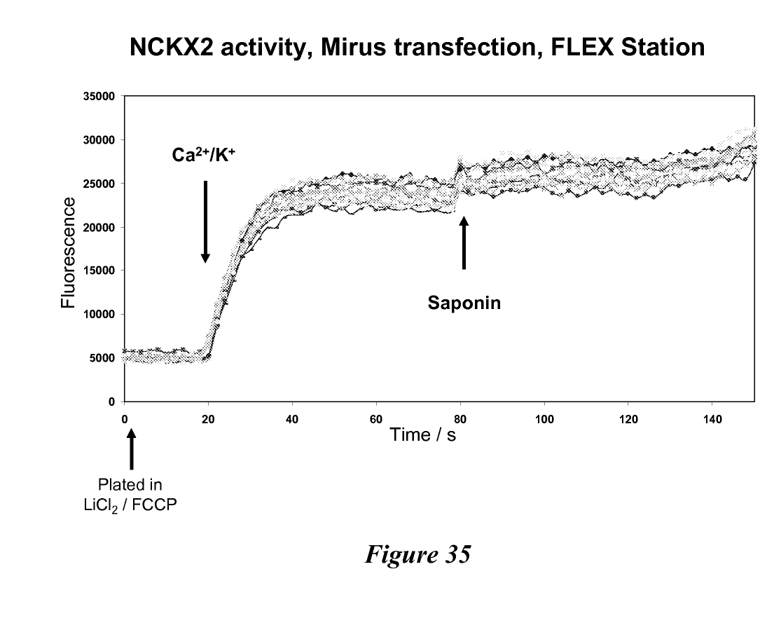

FIG. 35—NCKX2 assay. A high throughput NCKX2 assay of Minis transfected HEK 293 cells. Each trace on the graphs represents the signal from one well.

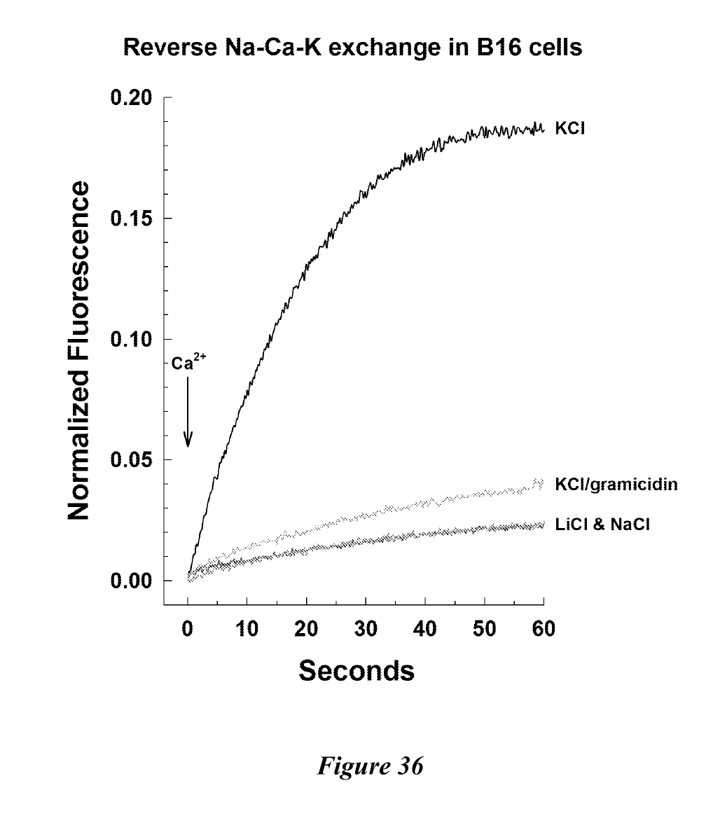

FIG. 36—B16 cell NCKX activity

Fluorescence detection of NCKX activity in B16 melanoma cells.

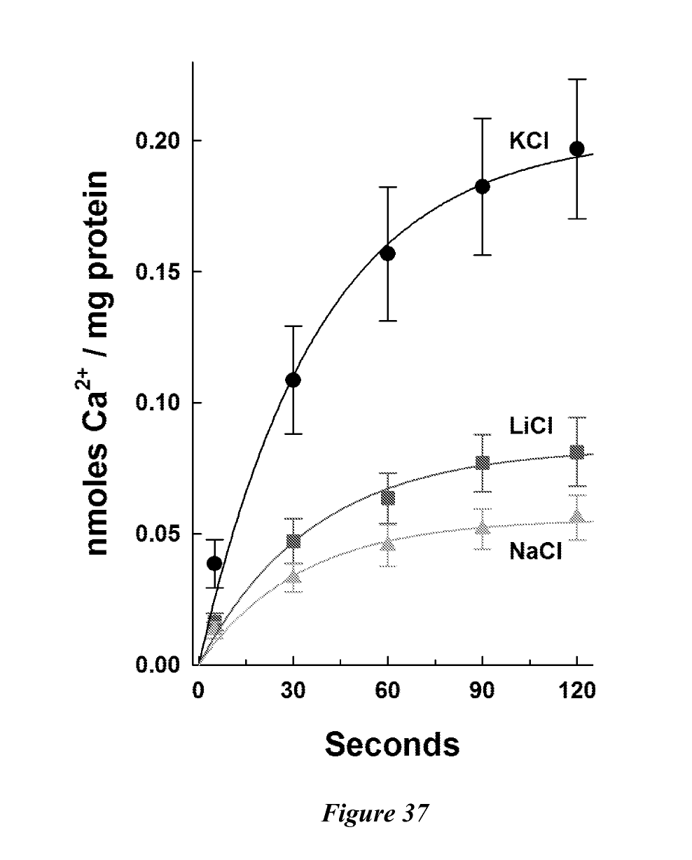

FIGS. 37—B16 cell NCKX activity

⁴⁵Ca detection of NCKX activity in B16 melanoma cells.

### EXAMPLE 1

### Distribution of NCKX5 in Individuals of S. Asian Ancestry

Studies have shown that in a sample of individuals of UK volunteers having South Asian ancestry there is a correlation between different allelic versions of NCKX5 and skin colour (US 2007/0148664), and similar results have been described by Sabeti et al (2007), Nature 449: 913-916.

NCKX5 has a single nucleotide polymorphism that encodes variation at amino acid position 111 and in dark skins there is a predominance of the Ala111 amino-acid residue, whilst in lighter skins there is a predominance of the Thr111 amino-acid residue. FIG. 2 shows the distribution of these two alleles in darker and lighter skin types, from a total of 230 volunteers of South Asian descent. Volunteers were selected by taking measurements of non-sun exposed skin using a chromameter. The L* reading of the chromameter gives a direct read out of the reflectance of skin, which in turn is directly related to the melanin content of the skin. Volunteers who fell into the 20% ‘extreme’ tails of the skin colour distribution were genotyped. These volunteers therefore had either lighter or darker skin colour compared to the average. The allele frequency difference for the non-synonymous polymorphism at amino acid 111 of NCKX5 is 39% with an extremely low chance of false discovery (False Discovery Rate=9.7×10⁻¹³).

### EXAMPLE 2

### Activity of NCKX5

The activity of NCKX5 and the effect of the two alleles (Ala111 and Thr111) on Sodium-Potassium/Calcium exchange was investigated by mutating an NCKX exchanger (NCKX2) that is naturally found inserted in retinal rod photoreceptor cell membranes to more closely resemble NCKX5. The methods used to produce these constructs are described in Winkfein et al (2003) Biochemistry 42 pp 543-552 and the methods used to investigate their effects are described by Kang et al (2005a).

By mutating residue 177, which is the position of NCKX2 which is equivalent to residue 111 of NCKX5, two forms of NCKX2 were produced that correspond to the NCKX5 SNP at residue 111, i.e. one NCKX2 form had the naturally occurring residue Ala177, while the other form had this residue mutated to Thr177. (See FIG. 3 for the position of the variant in the three dimensional structure of the protein and a comparison of the sequence identity of the NCKX family.)

The modified NCKX2 was expressed and the calcium exchange function of the molecule was measured. It was found that the Thr177 version that is associated with light skin showed a significantly reduced calcium exchange in comparison to the Ala177 version which is associated with dark skin (see FIG. 4).

### EXAMPLE 3

### NCKX5 Transcript and Localisation Analysis

### Transcript Expression

cDNA was derived from cultured melanocytes, fibroblasts and keratinocytes isolated from donors of Indian origin with various skin colours and also commercially sourced from Caucasian and Negroid donors.

The cDNA derived from Indian volunteers was derived from skin biopsies. Commercially sourced cDNA was derived from different human body tissues (brain, colon, kidney, lung, muscle, stomach and uterus).

NCKX pre-designed and validated Sybr primers were from Qiagen: 5196, 5197, 5198, 5199, 5200, 5201, 5202, 5203, 5204, 5205, 5206, 5207. The primers/probes used were: NCKX5 ABI primer/probe set: hs01385406_g1, spanning exons 3-4 FAM-linked, and huTBP ABI primer/probe set 4326322E, VIC-linked.

mRNA levels were measured using real-time PCR with Taqman® probes (purchased from Applied Biosystems) and normalised to housekeeper gene human transcription factor IID TATA box binding protein (huTBP).

NCKX5 mRNA transcript was detected in all the cultured melanocytes and skin biopsies tested. The NCKX5 mRNA expression levels in skin biopsies do not appear to correlate with colour differences, ethnic origin or SLC24A5 genotype in a small cohort tested (n=22)

NCKX5 mRNA was not detectable by real-time PCR using Taqman® probes in the non-skin tissues (brain, colon, kidney, lung, muscle, stomach, and uterus).

SLC24A5 mRNA was not detectable by real-time PCR using Taqman® probes in cultured fibroblasts or keratinocytes (i.e. skin cells other than melanocytes). However, evidence was obtained using Sybr green detection (a real-time PCR method using Qiagen quantitect kit using a Biorad icycler) that low levels of SLC24A5 mRNA may be present in cultures of dermal fibroblasts (at much lower levels than cultured melanocytes from the same donor).

In cultured melanocytes, NCKX5 mRNA levels are higher than NCKX1 (SLC24A1) mRNA levels. NCKX4 (SLC24A4) and NCKX6 (SLC24A6) mRNA is detectable in cultured melanocytes, but at lower levels than NCKX5 or NCKX1 (SLC24A1), using the Sybr green detection method.

### Protein Localisation

Rabbit polyclonal antisera were raised against 2 peptides fragments derived from NCKX5 (one in the predicted large hydrophilic loop and one in the carboxy-terminus of the protein). Antibodies were affinity purified against the peptides. The antibodies were generated by Eurogenetec using the following protocol: Primary immunisation of 2 rabbits with both peptide fragments followed by 3 subsequent boosted immunisations 2, 4 and 8 weeks after primary immunisation. Peptides were conjugated to keyhole limpet haemocyanin and the final bleed was collected 12 weeks after the initial immunisation. For affinity purification, each peptide was conjugated to Sepharose and 5 ml of final bleed sera was incubated with it in batch, packed into a column, washed in phosphate buffered saline (PBS) and eluted into 100 mM glycine-HCL pH2.5. The eluate was neutralised in 1M Tris at pH9 and buffer exchanged into PBSA.

Cultured primary human melanocytes from caucasian or negroid donors were grown on glass cover-slips and fixed in 2% paraformaldehyde and permeabilised with 0.5% Saponin.

In situ hybridisation was performed on the fixed cells using the anti-NCKX5 antibodies according to standard protocols. Secondary detection was conducted using donkey anti-rabbit Alexa-fluor 488 (Invitrogen). Visualisation of the bound antibodies was conducted using confocal fluorescence microscopy.

No evidence of plasma membrane staining indicated that NCKX5 was not localised in the plasma membrane.

Punctate staining was found throughout cell, indicating the presence of NCKX5 in the melanocyte (see FIG. 5, for staining)

### EXAMPLE 4

### Construction of NCKX5 Expression Vector

All clones were inserted between the XhoI/NotI DNA-restriction sites in either vectors pEIA and pcDNA3.1 The pIEA vector is available from Cytostore as the TriplExpress vector (www.cytostore.com) and its structure and sequence is shown in FIG. 6. The PcDNA3.1 vector may be purchased from Invitrogen (www.invitrogen.com) and its structure is shown in FIG. 7.

DNA (GAGTTT) encoding the NCKX5 amino acid sequence (amino acids 63-64, EF) was modified using standard recombinant methods to introduce an EcoRI DNA restriction site (GAATTC). The insertion of the EcoRI restriction site does not change the encoded amino acid but introduces a restriction site for a tag to be optionally inserted. In some clones the nucleic acid molecule coding for the myc tag protein sequence (QKLISEEDL) was inserted immediately upstream of the newly inserted EcoRI restriction site, i.e. in the region of the N terminus. The myc tag is recognised by a monoclonal antibody, which can be used in a Western blot to identify newly synthesised proteins. The nucleic acid molecule encoding the 1D4 tag was added immediately before the stop codon at the end of the NCKX5 sequence i.e. at the C terminus. The 1D4 tag is recognised by a monoclonal antibody, which can also be used in Western blot to identify the newly synthesised proteins.

The N-terminal sequence of hsNCKX2 from position 1 (M) to 120 (Q), plus a myc tag at position 84 of the WT sequence, was ligated to a partial construct of hsNCKX5 at amino acid 63 taking advantage of the EcoRI restriction site.

The sequences of the clones that were prepared as described above are given in FIGS. 8 to 15.

### EXAMPLE 5

### Expression of NCKX5 in Host Cells

Insect High Five™ cells were transfected with the vectors of Example 4, e.g. coding for the full length human NCKX5 protein coupled to the leader sequence coding for the human NCKX2 signal peptide, using the lepidopteron expression system method described by Farrell et al (1998) Bio/Technology 60 pp 656-663.

High Five™ cells were collected and washed twice with 150 mM NaCl, 20 mM Hepes (pH 7.4), 80 mM sucrose, and 200 mM EDTA. The final pellets were resuspended in 200 ml of ice-cold radioimmune precipitation buffer containing 1% Triton X-100, 0.5% deoxycholate, 140 mM NaCl, 25 mM Tris (pH 7.5), 100 mM EDTA, and a protease inhibitor tablet (Roche Molecular Biochemicals catalogue number 1 836 170), and incubated on ice for 20 min. The samples were spun down in a microcentrifuge for 5 min at 20,000 3 g. Supernatants were removed and assayed for protein concentration using the Bradford dye-binding procedure (Bio-Rad). Bovine serum albumin (BSA) was used as the standard in all protein assays.

Protein samples were separated on an 8% SDS-polyacrylamide gel and either stained with Gelcode Blue (Pierce) or transferred onto nitrocellulose (Bio-Rad) in 25 mM Tris buffer, pH 8.3, containing 192 mM glycine, 20% methanol, and 0.05% SDS. For Western blotting, the membranes were blocked for 1 h in TBST (10 mM Tris, pH 8.0, 100 mM NaCl, 0.05% Tween 20) and 10% skim milk, briefly rinsed in TBST, and subsequently incubated for 1 h at room temperature with primary antibody (1:20 dilution of PMe-1B3 or 10 mg/ml of 6H2 antibody) in TBST with 1% skim milk added. After washing, the membranes were incubated for 1 h with a 1:5,000 dilution of sheep anti-mouse immunoglobulin conjugated to horseradish peroxidase (Amersham Pharmacia Biotech) in TBST plus 1% skim and then washed again. Immunodetection was carried out using LumiGlo chemiluminescent reagents (New England Biolabs).

HEK 293 cells were cultured in Earle's minimum essential medium (EMEM) at 37° C., 5% CO₂ in T175 cm² cell culture flasks containing sodium pyruvate and non-essential amino acids purchased from Biowhittaker and also containing 10% foetal calf serum (FCS) and 2 mM L-glutamine purchased from Sigma.

HEK293 cells were transfected with a vector as made in Example 4e.g. coding for the truncated human NCKX5 protein coupled to the leader sequence coding for the human NCKX2 signal peptide, using the calcium phosphate precipitation transfection method described by Kang et al (2005a).

Results of the expression tests are shown in the Western blots of FIGS. 16 and 17. These blots show that the test cells expressed both NCKX5 and the NCKX2/NCKX5 chimera in their various tagged forms.

### EXAMPLE 6

### NCKX Activity Assay

Adherent HEK 293 cells plated in 10 cm dishes were transfected with hNCKX2 using calcium phosphate transfection for 48 h (Kang et al, 2005a). Following transfection, cells were trypsinised and re-suspended in Na-loading buffer containing 150 mM NaCl supplemented with ouabain and also containing Fluo4-AM calcium-sensitive dye, and placed on an orbital rotary mixer at room temperature for 35 min. Following dye loading, cells were washed once in Na-loading buffer supplemented with ouabain, resuspended in the same, diluted into a lithium chloride based assay buffer containing FCCP, and 40,000 cells per well (200,000 cells/ml) were placed into a 96 well glass-bottomed assay plate coated with a high binding capacity (600 ng/cm²) polystyrene resin (plates purchased from Greiner Bio-One). The plate was then centrifuged at 300 g for 60 sec so that the cells settled at the bottom of the well. Without allowing the cells to adhere to the plate, the assay plate was transferred to a Molecular Devices FLEXStation, and intracellular fluorescence (i.e., NCKX2 activity) was measured upon addition of 250 μM free Ca²⁺ and 40 mM K⁺ (in the form of CaCl₂ and KCl, respectively). At reaction plateau, saponin was added to cells to determine maximum fluorescence as a dye-loading control.

In FIG. 34, untransfected HEK 293 cells were also analysed as a negative control. A similarly high degree of reproducibility was obtained following transfection of HEK293 cells with Minis transfection reagent (FIG. 35). Each trace on the graphs represents the signal from one well.

When measuring the effect of test compounds on the calcium flux, the test compound is added to the test cells prior to the treatment with CaCl₂ and KCl. Compounds from a library can be added at various doses for various times at 37° C. For data analysis, the fluorescence of untreated control cells is subtracted from test cells. The amount of calcium flux is quantitated by comparison to a serially-diluted reference standard curve. The effect(s) of test compounds on the rate and/or amount of calcium flux compared to untreated control cells is determined and any increase or decrease in activity recorded. The assay can assess the effect of test compounds on maximal fluorescence, rate of fluorescence, Vₘₐₓ, Kₘ or time to maximum fluorescence compared to untreated cells. Test compounds which are determined to be exerting an effect can be re-tested over a broad concentration range to confirm efficacy. Additionally, this method can be used to assess calcium or potassium dependence by addition of varying calcium or potassium concentrations to the test cells; for example, a K⁺ concentration within 10-20 mM may be particularly useful. This experiment may include a co-transfection with a fluorophor reporter plasmid to control for transfection efficiency. The assay can be performed manually but can also be automated, for example using a Hamilton robotic system. Once transfection and plating are complete, the robot is able to perform all washing, dye loading, temperature-dependent incubation of plates, compound additions and transfer of plates to FLEXstation. For higher throughput screening, the assay may be modified for use in 384 well or higher multi well plates.

### EXAMPLE 7

### Investigation of NCKX Activity in Mouse B16 Cells

### Methods

All materials were purchased from Sigma except: B16 murine melanoma cells (ATCC); Fluo4-AM, Ribo Green (Molecular Probes); thapsigargin (Santa Cruz); Greiner black, glass-bottomed 96 well plates (Greiner Bio-One); Stealth™ RNAi duplexes, Lipofectamine 2000, Opti-MEM (Invitrogen); RNeasy total RNA extraction kits, QuantiTect PCR primers (Qiagen); first strand cDNA synthesis kits (Roche); ibidi μ-flow slides (ibidi Integrated Bio Diagnostics); SYBR Green PCR master mix (Bio-Rad);

B16 mouse melanoma cells were cultured in EMEM supplemented with 10% FCS and 2 mM L-glutamine at 37° C., 5% CO₂ in T175 cm² flasks and were sub-cultured twice weekly using trypsin-EDTA. HEK 293 cells were cultured in EMEM with 2 mM L-glutamine and Earle's BSS adjusted to contain 1.5 g/L sodium bicarbonate, 0.1 mM non-essential amino acids, and 1.0 mM sodium pyruvate, 90%; heat-inactivated horse serum, 10% 24 h prior to transfection, B16 cells were plated at 25,000 cells/well in a 48-well plate. Cells in quadruplicate wells were transfected with 2μg/ml lipofectamine 2000 and 50 nM Stealth™ RNAi duplexes targeting murine SLC24A5 or scrambled controls (from Invitrogen). Reactions were also performed with lipofectamine only or untreated negative controls. All dilutions were performed with Opti-MEM. RNAi duplex sequences were:

| duplex 370 | |
|---|---|
| antisense: | 3′-UGAAAGUUGCACCUGCAACAUCCUG-5′; |
| sense | 5′-CAGGAUGUUGCAGGUGCAACUUUCA-3′; |
| duplex370 scrambled control | |
| antisense: | 3′-UGACUAUUGACACGCGUCACAACUG-5′; |
| sense | 5′-CAGUUGUGACGCGUGUCAAUAGUCA-3′; |
| duplex 762 | |
| antisense: | 3′-UUCAAUUCUCUCCUCCAUAGCUCUG-5′ |
| sense | 5′-CAGAGCUAUGGAGGAGAGAAUUGAA-3′; |
| duplex 762 scrambled control | |
| antisense: | 3′-UUCUCACUUACUCUCCUCGAUACUG-5′; |
| sense | 5′-CAGUAUCGAGGAGAGUAAGUGAGAA-3′ |
| duplex 1265 | |
| antisense: | 3′-AUAUCAAACACAUUGGAUCCCACGA-5′ |
| sense | 5′-UCGUGGGAUCCAAUGUGUUUGAUAU-3′; |
| duplex 256 | |
| antisense | 3′-UAAUGAGGAAGUAGAUUACGAUACC-5′ |
| sense | 5′-GGUAUCGUAAUCUACUUCCUCAUUA-3′; |
| duplex 567 | |
| antisense | 3′-AUACACUGCACAGUCUCGGAAGAGG-5′ |
| sense | 5′-CCUCUUCCGAGACUGUGCAGUGUAU-3′. |

Cells were incubated with transfection reagents for 6-8 h, reagents were removed and replaced with phenol red-free DMEM supplemented with 10% FCS and 4 mM L-glutamine for 72 h. At 72 h, cells were taken for viability and total protein determination and supernatants analysed for melanin production.

SLC8 and SLC24 mRNA Analysis B16 Murine Melanoma Cells

Following trypsinisation, total RNA was extracted from B16 cells using the RNeasy mini kit, with on-column DNAse treatment, as per the manufacturer's instructions. Total RNA concentration of extracts was quantified using Ribo Green as per the manufacturer's instructions, with fluorescence measured with a BMG Fluostar Optima plate reader. Absorbance values for unknown samples were compared to a seven point reference curve with a dynamic range of 15 ng/μl-1 μg/μl.

First strand cDNA synthesis was performed from 1 μg total RNA by reverse transcription using the first strand synthesis kit, as per the manufacturer's instructions. RT was performed in 20 μl reactions and where more than 20 μl cDNA was required, multiple reactions were performed and cDNA pooled. SLC24 mRNA expression was then analysed by real-time PCR with SYBR Green detection using a Bio-Rad iCycler. QuantiTect PCR primers directed against exon-exon boundaries within murine SLC8A1-SLC8A3, murine SLC24A1-SLC24A6 and murine GAPDH were purchased from Qiagen. Primer efficiency was confirmed by serial dilution of cDNA and melt-curve analysis. Target gene expression was normalised against GAPDH mRNA expression using the Δ_{CT} method.

See table of FIG. 22 showing that NCKX5 (SLC24A5) is expressed in B16 cells but that SLC8 is not.

SLC8 and SLC24 mRNA Analysis Dark Human Melanocytes

Following trypsinisation, total RNA was extracted from dark human melanocytes using the RNeasy mini kit, with on-column DNAse treatment, as per the manufacturer's instructions. Total RNA concentration of extracts was quantified using Ribo Green as per the manufacturer's instructions, with fluorescence measured with a BMG Fluostar Optima plate reader. Absorbance values for unknown samples were compared to a seven point reference curve with a dynamic range of 15 ng/μl-1 μg/μl.

First strand cDNA synthesis was performed from 1 μg total RNA by reverse transcription using the first strand synthesis kit, as per the manufacturer's instructions. RT was performed in 20 μl reactions and where more than 20 μl cDNA was required, multiple reactions were performed and cDNA pooled. SLC24 mRNA expression was then analysed by real-time PCR with SYBR Green detection using a Bio-Rad iCycler. Primer sequences were:

| SLC24A1: | |
|---|---|
| forward: | 5′-TCTGCACAACAGCACCAT-3′; |
| reverse | 5′-CTCTCCTCCTCCTTCTCCTT-3′; |
| SLC24A2: | |
| forward: | 5′-ATGATACACACCCTTGACC-3′; |
| reverse | 5′-CCTTTTCTCTGAACCTCCCTT-3′; |
| SLC24A3: | |
| forward: | 5′-CGTCTTATACTTCACTGTACCC-3′; |
| reverse | 5′-AACCAATGATTGTGACCATCC-3′; |
| SLC24A4: | |
| forward: | 5′-GACACAGACAGCCAAGAA-3′; |
| reverse | 5′-GCATAGAACATATACAGAGCACCA-3′; |
| SLC24A5: | |
| forward: | 5′-GAGATGGAGGCATCATAATCTA-3′; |
| reverse | 5′-CCTGAGACAATCCAAGGGATTC-3′ |
| SLC24A6: | |
| forward: | 5′-AGGCTTCACTGGCTCTT-3′; |
| reverse | 5′-AGGCATCTCCAATGCTGTTC-3′; |
| GAPDH: | |
| forward: | 5′-GGACCTGACCTGCCGTCT-3′; |
| reverse: | 5′-TAGCCCAGGATGCCCTTG-3′. |

QuantiTect PCR primers directed against exon-exon boundaries within human SLC8A1-SLC8A3 were purchased from Qiagen. Primer efficiency was confirmed by serial dilution of cDNA and melt-curve analysis. Target gene expression was normalised against GAPDH mRNA expression using the Δ_{CT} method.

### Analysis of Intracellular NCKX Activity in Various Cells in Suspension

An assay for intracellular Na-induced Ca²⁺ release was developed based on that previously described (Altimimi & Schnetkamp, 2007). B16 cells were trypsinised and centrifuged at 300 g, 3 min. Cells were resuspended with 500 μl basal DMEM and 12 μM Fluo3-AM and incubated for 35 min at room temperature with rotation on an orbital rotary mixer. Cells were centrifuged 300 g, 2 min and washed in 1.5 ml Na⁺ loading media. Cells were resuspended in 275 μl Na⁺-loading buffer (150 mM NaCl, 20 mM Hepes pH 7.4 with arginine, 3 mM KCl, 1.5 mM CaCl₂, 10 mM glucose, 25004 sulfinpyrazone). 50 μl B16 cells were suspended in 2 ml KCl media in a plastic, clear on all sides, cuvette. A magnetic flea was placed in the cuvette and the cuvette placed in a luminescence spectrometer with magnetic stirrer enabled and jacket heated to 25° C. The trace was started with readings every 1 second. At 10 seconds 204 FCCP, 104 gramicidin and 104 thapsigargin was added. At 180 seconds, 75 mM NaCl was added, at 200 seconds 350 μM CaCl₂ was added and once the trace had reached plateau, 0.01% saponin was added. The mean fluorescence counts of the 10 seconds immediately prior to sodium addition were used to subtract background signal from each of the data points of the sodium-induced calcium trace. The mean fluorescence counts for the final 10 seconds of the saponin-induced trace were then used to normalise each data point of the sodium-induced calcium trace. FIG. 18 shows the dose-dependent nature of Na⁺ dependent Ca²⁺ release in B16 cells when studied in cuvette suspension.

### Analysis of Intracellular NCKX Activity in Various Cells by HTS Method

The suspension assay previously described was modified for use in 96 well formats (Altimimi & Schnetkamp, 2007). Cells were plated into Greiner glass bottomed 96 well plates at 100,000 cells/well and adhered overnight. Media was removed and cells loaded with the Ca²⁺-sensitive dye Fluo4-AM in serum-free media at 37° C. for 30 min. Cells were washed with sodium-loading buffer (150 mM NaCl, 20 mM Hepes pH 7.4 with arginine, 3 mM KCl, 1.5 mM CaCl₂, 10 mM glucose, 250 μM sulfinpyrazone) and following washing, 30 μl sodium-loading buffer placed onto cells. 100 μl of KCl assay buffer (150 mM KCl, 20 mM Hepes pH 7.4 with arginine, 100 μM EDTA) containing 2 μM FCCP, 1μM gramicidin and 1 μM thapsigargin was placed onto cells and the plate inserted into a Molecular Devices FLEXStation. Fluorescence readings were taken every 3 seconds for 300 seconds. 120 mM NaCl was added at 120 seconds, 350 μM CaCl₂ was added at 200 seconds and 0.01% saponin added at 260 seconds. Following analysis, data was transferred to Excel. Background fluorescence was subtracted from each Na⁺ and saponin-induced fluorescence components. Background subtracted Na⁺ fluorescence was then normalised to background subtracted saponin fluorescence by multiplication of Na⁺ data by the reciprocal of saponin data.

The suitability of this assay for HTS was assessed by analysis of the Z′ factor, signal to noise ratio and signal to background ratio. One plate of B16 cells was analysed on each of three separate days as described above. The Z′ factor was calculated as:

{1−[(3*agonist SD)+(3*NSB SD)]}/(agonist mean−NSB mean) as previously described (Chen 2006, Zhang 1999) where NSB is non-specific background. The Z′ factor for this assay was 0.4, S:N 22.5, S:B 2.8, suggesting that the assay was suitable for HTS. This assay format was automated for use on a Hamilton robotic platform for HTS.

FIGS. 19 and 20 show the dose dependent nature of Na⁺ dependent Ca²⁺ release in B16 cells when studied in 96 well assays that simulate high throughput assays.

### Analysis of Intracellular NCKX Activity by Real-Time Confocal Microscopy

The assay described was adapted for use by real-time confocal microscopy on a Leica TCS SP1 confocal microscope. Cells were seeded into ibidi μ-flow slides and adhered overnight. Media was removed and cells loaded with Fluo4-AM in serum-free DMEM at 37° C. for 30 min. Cells were washed with sodium-loading buffer (150 mM NaCl, 20 mM Hepes pH 7.4 with arginine, 3 mM KCl, 1.5 mM CaCl₂, 10 mM glucose, 250 μM sulfinpyrazone) and following washing the slide was placed on the stage of the Leica TCS SP1 Confocal Scanning Laser Microscope (Leica Microsystems GmbH, Wetzlar, Germany). The scanning head was fitted to an inverted Leica DM IRBE microscope. A 40× plan apo 1.25 n.a. oil immersion phase contrast objective was used for collecting the images simultaneously in fluorescence and phase contrast. For image acquisition a frame size of 512×512 pixels was chosen and the sample scan rate was set to either maximum (1 frame every 0.87 seconds) or more typically one frame every 2 seconds. An argon ion laser with an excitation wavelength of 488 nm was used to excite the Fluo-4-loaded cells. Fluorescence emission was captured from 500-585 nm. The field of view in all images was 250×250 μm. Typical datasets were collected over 5 minutes. At time zero the cells were switched to KCl assay buffer (150 mM KCl, 20 mM Hepes pH 7.4 with arginine, 100 μM EDTA containing 2 μM FCCP, 1 μM gramicidin and 1μM thapsigargin). At 180 seconds, cells were switched to 75 mM NaCl and at 250 seconds cells were switched to 350 μM CaCl₂ buffer. Analysis of the images and the export of .avi movie files were performed via Leica LCS operating software and the results exported as .xml files. Pixel intensity values over time were assessed by highlighting cells within the image and plotting these as a function of time.

FIG. 21 demonstrates the confocal microscopy approach showing Na⁺ dependent Ca²⁺ release in B16 cells.

### Analysis of Heterologous NCKX2 Activity in HEK 293 Cells

Cultured HEK 293 cells were plated at 10,000 cells per well in glass bottomed 96 well plates and adhered overnight. Following adherence, cells were transfected with the short splice variant of the hNCKX2 gene cloned into pcDNA3.1 expression vector. The short splice variant lacks a stretch of 17 amino acids within the cytoplasmic loop of the protein. A c-Myc tag was also inserted at the BstE II site between bases 241-242, corresponding to amino acid residue 81 (Prinsen et al 2000, Winkfein et al 2003). Transfections were performed with Mirus 293 reagent as per the manufacturer's instructions for 48 h. Following transfection, media was removed and cells loaded with Fluo4-AM in serum-free DMEM at 37° C. for 30 min. Cells were washed with sodium-loading buffer (150 mM NaCl, 20 mM Hepes pH 7.4 with arginine, 3 mM KCl, 1.5 mM CaCl₂, 10 mM glucose, 250 μM sulfinpyrazone) and following washing, 30 μl sodium-loading buffer placed onto cells. 100 μl of KCl assay buffer (150 mM KCl, 20 mM Hepes pH 7.4 with arginine, 100 μM EDTA) containing 2 μM FCCP, 1μM gramicidin and 1μM thapsigargin was placed onto cells and the plate inserted into a Molecular Devices FLEXStation. Fluorescence readings were taken every 3 seconds for 500 seconds. 350 μM CaCl₂ was added at 150 seconds, 75 mM NaCl was added at 180 seconds and 0.01% saponin added at 350 seconds. Alternatively, HEK 293 cells heterologously expressing hNHCX2 were loaded with Fluo4-AM for 30 min at 37° C. in a physiological salt solution (150 mM NaCl, 20 mM HEPES pH 7.4 with arginine, 6 mM glucose, 0.25 mM sulfinpyrazone, 0.104 ouabain±1.5 mM CaCl₂). Following removal of extracellular dye, cells were switched to LiCl₂ assay buffer (150 mM LiCl₂, 20 mM HEPES pH 7.4 with arginine, 6 mM glucose, 0.1 mM EDTA). At 120 seconds, 350 μM CaCl₂ was added followed by 50 mM KCl to initiate maximal rate of reverse calcium exchange. At maximal fluorescence 0.01% saponin was added. Following analysis, data was transferred to Excel. Background fluorescence was subtracted from each Na⁺ and saponin-induced fluorescence components. Background subtracted Na⁺ fluorescence was then normalised to background subtracted saponin fluorescence by multiplication of Na⁺ data by the reciprocal of saponin data (see FIG. 23).

### REFERENCES FOR EXAMPLE 7

Altimimi & Schnetkamp (2007), J Biol Chem 282(6): 3720-3729
Chen et al Development of FLIPR-based HTS Assay for Gi-Coupled GPCRs. In Handbook of Assay Development in Drug Discovery, pages 305-317. Published by Taylor Francis. 2006.
Zhang et al (1999) A Simple Statistical Parameter for Use in Evaluation and Validation of High Throughput Screening Assays. J Biomol Screen 4(2): 67-73.
Prinsen et al (2000) Molecular cloning and functional expression of the potassium-dependent sodium-calcium exchanger from human and chicken retinal cone photoreceptors. J Neurosci 20:1424-1434.
Winkfein et al (2003) Scanning mutagenesis of the alpha repeats and of the transmembrane acidic residues of the human retinal cone Na⁺/Ca²⁺—K⁺ exchanger. Biochemistry 42:543-552.

### EXAMPLE 8

### Methods for the Assessment of NCKX5 Activity in Relation to Melanogenesis in B16 Cells

### A) Quantification of Melanin Production, Protein Content and Cell Viability of Cultured B16 Mouse Melanocytes.

### 1) Cell Treatments 72 Hours Post-Knockdown:

The phenol free DMEM culture media was removed from each well for quantification of secreted melanin. Each well of the culture plate was then rinsed once with Dulbecco's phosphate buffered saline (dPBS) (Sigma-Aldrich, Poole, UK) and replaced with 0.5 ml Wst1 reagent (diluted 1/10 in media) (Roche Diagnostics Ltd, West Sussex, UK). The culture plate was incubated at 37° C., 5% CO₂ for 30 minutes. The Wst1 reagent was removed from each well for determination of cell viability and each well was again rinsed with dPBS. 200 μl of 1% w/v Triton X-100 (Sigma-Aldrich, Poole, UK) in dPBS was added to each well of the culture plate. The plate was incubated for 20 minutes at 4° C. on an orbital shaker. Supernatant from each well was removed and centrifuged at 13000 g for 5 minutes to remove cell debris. Soluble fractions were kept for protein quantification.

### 2) Melanin Assay:

1 mg synthetic melanin (Sigma-Aldrich, Poole, UK) was dissolved in 50 μl of 100% DMSO. 950 μl phenol free DMEM+10% FCS was added drop-wise to create a 1 mg/ml top standard. This standard was serially diluted (5-fold) in media+FCS to give a standard curve for quantification of melanin in sample preparations (range from 1000 to 8 μg/ml). 100 ul each standard and sample was placed in a 96-well microtitre plate in duplicate (Greiner Bio-One Ltd, Gloucestershire, UK) and the optical density (OD) of each sample replicate was measured at 450 nm using a Dynex MRX plate reader (Dynex Technologies Ltd, West Sussex, UK). The melanin content of each culture fraction was calculated from the synthetic melanin standard curve.

### 3) Wst1 Assay:

The OD⁴⁵⁰ of 100 μl fractions (measured in duplicate) of post-incubation Wst1 reagent was measured. The OD⁴⁵⁰ of each treatment was compared to evaluate relative viability.

### 4) Protein Content Determination:

The protein content of each triton X-100 cell culture fraction was measured using a BCA assay kit (Perbio Science UK Ltd, Northumberland, UK) as per the manufacturer's instructions with the following modification: −10 μl of each solubilised protein fraction was placed in a 96-well microtitre plate in duplicate. 15 μl dH2O was added to each sample prior to the addition of the BCA colour reagent. A standard curve was prepared by 2-fold serial dilution (using 1% triton X-100 in dPBS) of BSA (range=2000 to 15.6 μg/ml). The plate containing all samples and standard solutions was incubated for 15 minutes and the OD⁵⁹⁵ was measured. Protein content of each sample was calculated from the BSA standard curve.

### 5) Calculation of μg Melanin Per μg Protein:

The μg/ml melanin content for each sample was divided by 2 to give the total melanin per sample. The μg/ml protein content derived from the BCA assay was multiplied by 2.5 (to account for the assay dilution step, then divided by 5 (accounting for the volume of triton used to lyse the cells and release protein. Finally, for each treatment, the total melanin (μg) value was divided by the total protein (μg) value to give μg melanin per μg protein.

See FIG. 29 for results showing that NCKX5 protein does modulate pigment production.

### B) Quantification of Melanin Production, NCKX5 Protein Expression and Cell Viability of Cultured Human Melanocytes.

After siRNA-mediated knockdown of SLC24A5, the following assays were conducted:

### 1) Cell Viability Assessment:

Melanocyte viability 5 days after siRNA treatment was assessed by Wst1 assay as described in A) but with the following modifications: 1 ml of 1/10 diluted (in culture media) Wst1 reagent was added to each well of 6-well culture plates containing human melanocytes. The plates were incubated for 60 minutes at 37° C., 5% CO² prior to the removal and OD⁴⁵⁰ assessment of the reagent.

### 2) Protein and Melanin Fractionation:

Cultured Cells (trypsinised off of 6-well culture plates) were lysed on ice for 20 minutes in 1.5 ml eppendorfs using 100 μl per sample of 1% triton X-100 in dPBS containing protease inhibitor cocktail (Sigma-Aldrich, Poole, UK). Cell extracts were centrifuged (10 minutes at 13000 g) to separate melanin and cell debris from the solubilised protein. The protein concentration of each supernatant fraction was determined by BCA assay as described in A).

### 3) SDS-PAGE and Electrophoretic Transfer to PVDF Membranes:

20 μg protein (as determined by BCA assay) was reconstituted into 20 μl of 1×LDS loading buffer (Invitrogen Ltd, Paisley, UK) containing 1× reducing agent (Invitrogen Ltd, Paisley, UK), heated to 40° C. for 30 minutes and loaded onto 10% Novex bis-tris acrylamide gels with 1×MOPS running buffer (Invitrogen Ltd, Paisley, UK). Kaleidoscope molecular weight markers (Bio-Rad Laboratories Ltd, Hemel Hempstead, UK) were run alongside samples for size determinations. Protein was transferred onto PVDF membrane by electrophoresis transfer, using a Bio-Rad mini-cell II trans-blotter and 1×Tris-Glycine transfer buffer (Invitrogen Ltd, Paisley, UK)+15% v/v methanol (100V for 1 hour).

### 4) Western Blotting to Detect NCKX5:

Membranes containing transferred protein were “blocked” using 2% w/v skimmed milk protein (SMP) in PBS+0.05% tween20 (PBST) for 1 hour with gentle agitation. Peptide affinity purified rabbit polyclonal antibody (raised using a peptide equivalent to the C-terminus of NCKX5—GNNKIRGCGG) was diluted to 0.5 μg/ml using 2% SMP in PBST. The diluted antibody was incubated with the membrane for 2 hours at room temperature. The membrane was then rinsed with PBST (4×5 minutes washes). Peroxidase conjugated anti-rabbit IgG (Jackson ImmunoResearch Laboratories Inc. PA, USA) was diluted 1/4000 using 2% SMP in PBST and was incubated with the membranes for 1 hour. Finally, membranes were washed using 6×5 minute rinses with PBST. SuperSignal Western pico (Perbio Science UK Ltd, Northumberland, UK) chemiluminescence detection reagents were used to probe the membranes for secondary antibody binding as per the manufacturers instructions. Visualisation of results was via a Chemidoc XRS imaging system (Bio-Rad Laboratories Ltd, Hemel Hempstead, UK).

### 5) Measurement of Melanin Content:

To each pellet of melanin+cell debris obtained in section 2), 0.5 ml of diethyl ether:ethanol (1:1 ratio) was added. Eppendorfs were vortexed vigorously for 30 seconds and centrifuged at 13000 g for 5 minutes. The ether layer was carefully removed to waste and a further 0.5 ml was added. The tubes were agitated and centrifuged as before. The liquid phase was again removed and the melanin pellets were allowed to air dry at room temperature for 10 minutes. Melanin pellets were solubilised by the addition of 200 μl of 1M NaOH+10% DMSO and incubation (with occasional agitation) at 50° C. for 1 hour. 80 μl aliquots of each sample were transferred to a 96-well microtitre plate in duplicate. OD⁴⁵0 of each sample was determined and the melanin content of each fraction was calculated from a synthetic melanin standard curve, prepared as described above. In treatment comparisons, results were expressed as μg melanin per μg protein.

See FIGS. 30 and 31 for results showing siRNA knockdown demonstrating a reduction of SLC24A5 mRNA in mouse B16 cells and protein content results.

### EXAMPLE 9

### siRNA Knockdown Results in Human Cells

### Cell Culture

Primary human melanocytes isolated from lightly pigmented or darkly pigmented neonatal foreskin were obtained from Cascade Biologics. Melanocyte Growth Medium (MGM) refers to Medium 254 (Cascade Biologics) supplemented with HMGS (Cascade Biologics). Melanocyte cultures were maintained in MGM at 37° C. with 10% CO₂. Cells were seeded at either 2×10⁴ cells/cm² (melanogenesis experiments) or 1×10⁴ cells/cm² (immunofluorescence experiments) and allowed to attach for 24 h prior to transfection.

Transfection of Cells with Oligonucleotides

Stealth™ siRNA duplex oligonucleotides were purchased from Invitrogen and used at a final concentration of 20-100 nM. A scrambled, non-targeting version of duplex 260 was used as a control.

TABLE
Sequences and exon targets of siRNA duplexes
designed against human SLC24A5

| siRNA | | Exon |
|---|---|---|
| duplex | Sequence | target |
| 185 | 5′ AGGGCCACAGGAAATAGCACCCAAT 3′ | 1&2 |
| | 3′ TCCCGGTGTCCTTTATCGTGGGTTA 5′ | boundary |
| 260 | 5′ GAGCGCAGAGATGGAGGCATCATAA 3′ | exon 2 |
| | 3′ CTCGCGTCTCTACCTCCGTAGTATT 5′ | |
| 301 | 5′ CGTTTACATGTTCATGGCCATATCT 3′ | exon 2 |
| | 3′ GCAAATGTACAAGTACCGGTATAGA 5′ | |
| 492 | 5′ GCACCATCCTTGGATCTGCAATTTA 3′ | 3 and 4 |
| | 3′ CGTGGTAGGAACCTAGACGTTAAAT 5′ | boundary |
| 1110 | 5′ CCGCATTTACATATATCCTGGTTTG 3′ | exon 7 |
| | 3′ GGCGTAAATGTATATAGGACCAAAC 5′ | |

Lipofectamine™ 2000 (Invitrogen) was diluted (1:50) in Opti-MEM® I Reduced Serum Medium (Gibco) and incubated at room temperature for 15 min. siRNA duplexes were diluted in Opti-MEM® I and combined with 1 volume of diluted Lipofectamine™ 2000. Following a 15 min incubation period at room temperature, 4 volumes of Opti-MEM® I was added, and the resultant mixture used to transfect the cells. Following a 6-8 h incubation period at 37° C. with 10% CO₂, the cells were transferred to MGM. Cells were transfected on day 0 and again on day 5 if required.

### Immuno Fluorescence

Cells were grown on glass coverslips and transfected with siRNA duplexes where indicated. Following a 72 h, 5 day or 10 day incubation, cells on coverslips were washed twice with PBS then fixed with 2% PFA in PBS for 20 min, washed a further 3 times, and permeabilised with 0.5% saponin in PBS. Coverslips were incubated with 0.2% BSA/0.1% saponin/PBS for 1 h at room temperature, then incubated with primary antibodies in 0.2% BSA/0.1% saponin/PBS for 1.5 h at room temperature. Primary antibody dilutions used were anti-NCKX5(827), 1:500; anti-NCKX5(826), 1:25 and anti-TGN46 (AbD Serotec), 1:200. Coverslips were washed twice with 0.1% saponin/PBS and twice with 0.2% BSA/0.1% saponin/PBS, then incubated with secondary antibodies in 0.2% BSA/0.1% saponin/PBS for 45 min at room temperature. Secondary antibodies (Alexa 488-conjugated anti-rabbit or Alexa 633-conjugated anti-sheep) were purchased from Invitrogen and used at a dilution of 1:500. Coverslips were washed twice with 0.2% BSA/0.1% saponin/PBS and twice with 0.1% saponin/PBS, then rinsed with Milli Q and mounted with VectaShield (Vector Laboratories) mounting medium. Cells were observed and photographed using a confocal microscope.

See FIG. 24 for results of SLC24A5 reduction over time by siRNA duplexes. See FIG. 25 for visual interpretation of the reduction of melanin pigment in human melanocytes after knockdown when compared to non-knockdown results. See FIG. 26 for Immunofluorescence results. And also see FIG. 27 for quantitative analysis results showing melanin production reduced in knockdown siRNA treated cells.

### EXAMPLE 10

### Demonstration of Lack of Native SLC24A5 (NCKX5) in HEK 293 Cells

### Sodium-Induced Intracellular Calcium Release in Various Cells

We have demonstrated that SLC24A5 mRNA is undetectable in HEK 293 cells and human keratinocytes. Moreover, transcript expression is lower in MEWO cells than primary human melanocytes or B16 cells. Therefore we investigated the extent of Na+-induced intracellular Ca²⁺ release in these cells. 100,000 cells were plated in triplicate to a 96 well plate, adhered overnight and intracellular Ca²⁺ release investigated as described in Example 7, section headed “Analysis of intracellular NCKX activity in various cells by HTS method”. Whilst some activity was detected in HEK 293, MEWO and keratinocytes, the rank order of normalised activity follows our prediction based on transcript expression profiles (FIG. 32).

The early time-points from the trace on FIG. 33 show a clear difference in rates of sodium-induced calcium release from an intra-cellular store between cells expressing high levels of SLC24A5 transcript (dark and light melanocytes, B16 melanocytes) and those with undetectable SLC24A5 transcript (HEK-293 and keratinocytes). The MeWo cells have intermediate levels of SLC24A5 transcript (and do not produce melanin under these conditions) which corresponds with an intermediate level of sodium-induced calcium release.

SLC24 and SLC8 mRNA Expression HEK 293 and Human Keratinocytes

To assess if SLC24A5 and SLC8 family members are expressed at the transcript level in HEK 293 cells, RNA was extracted from cultured HEK 293 cells and real-time PCR performed. SLC24A5 mRNA was undetected and SLC8 mRNA was detected at low levels. Previous studies have demonstrated that plasma membrane NCKX activity is undetected in untransfected HEK 293 cells suggesting that NCKX1-NCKX4 proteins are not expressed at detectable levels (Kang et al 2005a; Cooper et al 1999; Visser et al 2007).

| Gene | C_{T} ± SD |
|---|---|
| SLC24A5 | Not detected |
| SLC8A1 | 31.7 ± 0.2 |
| SLC8A2 | 30.6 ± 0.2 |
| SLC8A3 | 31.5 ± 0.2 |

The Table above shows 1 μg RNA extracted from HEK293 cells was reverse-transcribed and real-time PCR performed as described in the methods. Data are mean±SD triplicate reactions.

| Gene | C_{T} ± SD |
|---|---|
| SLC24A1 | 27.4 ± 0.2 |
| SLC24A2 | 36.1 ± 0.5 |
| SLC24A3 | 29.8 ± 0.0 |
| SLC24A4 | Not detected |
| SLC24A5 | 34.2 ± 0.6 |
| SLC24A6 | 24.0 ± 0.0 |
| SLC8A1 | 31.8 ± 0.2 |
| SLC8A2 | 33.8 ± 0.3 |
| SLC8A3 | Not detected |
| SNARE | 21.2 ± 0.1 |

The Table above shows mRNA extracted from human keratinocytes detected using SYBR green realtime PCR following reverse transcription of 1 μg of RNA. Table values are mean±SD duplicate reactions.

### REFERENCES FOR EXAMPLE 10

Kang et al (2005a) “Residues Contributing to the Calcium and Potassium Binding Pocket of the NCKX2 Na⁺/Ca²⁺—K⁺ Exchanger”. J Biol Chem 280(8): 6823-6833.
Cooper et al (1999). cDNA Cloning and Functional Expression of the Dolphin Retinal Sodium-calcium-Potassium Exchanger NCKX1: Comparison with the Functionally Silent Bovine NCKX1. Biochemistry 38: 6276-6283.
Visser et al (2007). Exchangers NCKX2, NCKX3, and NCKX4: Identification of Thr-551 as a Key Residue in Defining the Apparent K⁺Affinity of NCKX2. J Biol Chem 282(7): 4453-62.

### EXAMPLE 11

### Expression of NCKX5 in Follicular Melanocytes

We have analysed the expression of NCKX5 mRNA in follicular melanocytes using real-time PCR (using the methods described above). Briefly, 1 μg total RNA from follicular melanocytes from a female donor was reverse-transcribed using a First Strand cDNA synthesis Kit (Roche) according to the manufacturer's instructions. Reverse-transcription was performed in 20 μl reactions. NCKX5 mRNA expression was then analysed by real-time PCR with SYBR Green detection using a Bio-Rad iCycler. Primer sequences were:

| | |
|---|---|
| Forward: | 5′-GAGATGGAGGCATCATAATCTA-3′; |
| Reverse | 5′-CCTGAGACAATCCAAGGGATTC-3′. |

A cycle threshold (CT) value of 20.0 was obtained (data not shown), demonstrating that the NCKX5 transcript is expressed in follicular melanocytes.

### EXAMPLE 12

### NCKX Activity in B16 Melanoma Cells

### Fluo4 Experiments

B16 cells were lifted from one T150 flask, rinsed and resuspended in 500 μL of medium A containing 150 mM NaCl, 20 mM Hepes (pH 7.4), 20 μM CaCl₂, 150 μM EGTA, 0.25 mM sulfinpyrazone and 0.4 mM ouabain. 10 μM Fluo4-AM was added and the cells were incubated for 30 minutes. 2 μM FCCP and 1 μM thapsigargin were added during the last five minutes of the incubation. Cells were washed twice with 1 mL of medium A and resuspended in 500 μL, of medium A. 50 μL aliquots of the dye-loaded B16 cells suspension were diluted in a transparent plastic cuvette containing 1.9 mL of 150 mM LiCl, KCl or NaCl (as indicated), 20 mM Hepes (pH 7.4) and 0.1 mM EDTA. The cuvette is placed in a Series 2 luminometer (SLM instruments) and Fluo4 fluorescence is measured under continuous stirring as described (Altimimi & Schnetkamp, J. Biol. Chem. 282: 3720-29, 2007). Ca²⁺ influx is initiated at time zero by addition of 0.2 mM CaCl₂ and the increase in cytosolic free is monitored by an increase in fluorescence. Fluorescence is normalized to total fluorescence observed after permeabilizing the B16 cells through the addition of 0.025% saponin. In the trace labeled “KCl/gramicidin”, 5 μM gramicicin was added to the cuvette 2 minutes before addition of CaCl₂.

Results are shown in FIG. 36, demonstrating native NCKX activity in mouse melanoma B16 cells.

### ⁴⁵Ca Experiments

B16 cells were lifted from T150 flask, rinsed and resuspended in 500 μL, of medium A containing 150 mM NaCl, 20 mM Hepes (pH 7.4) 20 μM CaCl₂, 150 μM EGTA, and 0.4 mM ouabain. Cells were incubated in this medium for 30 minutes, spun down and resuspended in 280 μL, 150 mM LiCl, 20 mM Hepes (pH 7.4), 15 μM EDTA, 2 μM FCCP. 70 μL, cells were diluted to a final volume of 350 μL, in a medium containing 150 mM KCl, LiCl or NaCl (as indicated), 20 mM Hepes (pH 7.4), 35 μM CaCl₂ and 1 μCi ⁴⁵Ca. Time-dependent ⁴⁵Ca uptake was measured as described (Szerencsei et al., J. Biol. Chem. 275:669-76, 2000).

Results are shown in FIG. 37, demonstrating native NCKX activity in mouse melanoma B16 cells.

### EXAMPLE 13

### Pharmaceutical and Cosmetic Formulations

A further aspect of the invention provides a pharmaceutical formulation comprising a compound identified in the screening methods of the first aspect of the invention in admixture with a pharmaceutically or veterinarily acceptable adjuvant, diluent or carrier.

A still aspect of the invention provides a cosmetic formulation comprising a compound identified in the screening methods of the first aspect of the invention in admixture with a cosmetically acceptable adjuvant, diluent or carrier.

Preferably, the pharmaceutical formulation is a unit dosage containing a daily dose or unit, daily sub-dose or an appropriate fraction thereof, of the active ingredient, i.e. the compound identified in the screening methods of the first aspect of the invention.

The compounds may be administered in the form of a pharmaceutical formulation comprising the active ingredient identified in the screening methods of the first aspect of the invention, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds identified by the invention can be administered alone but will generally be administered in admixture with a suitable pharmaceutical excipient diluent or carrier selected with regard to the intended route of administration and standard pharmaceutical practice.

The compounds are typically administered topically in the form of a lotion, solution, cream, ointment or dusting powder. The compounds of the invention may also be transdermally administered, for example, by the use of a skin patch. For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Generally, in humans, topical administration of the compounds is the preferred route, being the most convenient. For veterinary use, a compound of the invention is administered as a suitably acceptable formulation in accordance with normal veterinary practice and the veterinary surgeon will determine the dosing regimen and route of administration which will be most appropriate for a particular animal.

### Dermatologically Acceptable Vehicle

The composition used according to the invention typically also comprises a dermatologically/cosmetically acceptable vehicle to act as a diluant, dispersant or carrier for the active components, i.e., the compounds identified in the screening methods of the first aspect of the invention. The vehicle may comprise materials commonly employed in skin care products such as water, liquid or solid emollients, silicone oils, emulsifiers, solvents, humectants, thickeners, powders, propellants and the like.

The vehicle will usually form from 5% to 99.9%, preferably from 25% to 80% by weight of the composition, and can, in the absence of other cosmetic adjuncts, form the balance of the composition.

### Optional Skin Benefit Materials and Cosmetic Adjuncts

Besides the actives active components, other specific skin-benefit actives such as sunscreens, skin-lightening agents, skin tanning agents may also be included. The vehicle may also further include adjuncts such as antioxidants, perfumes, opacifiers, preservatives, colourants and buffers.

### Product Preparation, Form, Use and Packaging

To prepare the topical composition, the usual manner for preparing skin care products may be employed. The active components are generally incorporated in a dermatologically/cosmetically acceptable carrier in conventional manner. The active components can suitably first be dissolved or dispersed in a portion of the water or another solvent or liquid to be incorporated in the composition. The preferred compositions are oil-in-water or water-in-oil or water-in-oil-in-water emulsions.

The composition may be in the form of conventional skin-care products such as a cream, gel or lotion, capsules or the like. The composition can also be in the form of a so-called “wash-off” product e.g. a bath or shower gel, possibly containing a delivery system for the actives to promote adherence to the skin during rinsing. Most preferably the product is a “leave-on” product; a product to be applied to the skin without a deliberate rinsing step soon after its application to the skin.

The composition may packaged in any suitable manner such as in a jar, a bottle, tube, roll-ball, or the like, in the conventional manner.

The method of the present invention may be carried out one or more times daily to the skin which requires treatment. The improvement in skin appearance will usually become visible after 3 to 6 months, depending on skin condition, the concentration of the active components used in the inventive method, the amount of composition used and the frequency with which it is applied. In general, a small quantity of the composition, for example from 0.1 to 5 ml is applied to the skin from a suitable container or applicator and spread over and/or rubbed into the skin using the hands or fingers or a suitable device. A rinsing step may optionally follow depending on whether the composition is formulated as a “leave-on” or a “rinse-off” product.

The formulation below describes an oil-in-water cream suitable for administering an active component identified in the screening methods of the first aspect of the invention. The percentages indicated are by weight of the composition.

| | Wt % | Wt % | Wt % |
|---|---|---|---|
| Mineral Oil | 4 | 4 | 4 |
| Petroselinic acid (triglyceride) ex Elysion | 1.15 | 2 | 3 |
| Green Tea Polyphenols | 0 | 2 | 0 |
| EGCG | 0 | 0 | 1 |
| Quercetin | 0.5 | 0 | 0 |
| Brij 56* | 4 | 4 | 4 |
| Alfol 16RD* | 4 | 4 | 4 |
| Triethanolamine | 0.75 | 0.75 | 0.75 |
| Butane-1,3-diol | 3 | 3 | 3 |
| Xanthan gum | 0.3 | 0.3 | 0.3 |
| Perfume | qs | qs | qs |
| Butylated hydroxy toluene | 0.01 | 0.01 | 0.01 |
| Water | to 100 | to 100 | to 100 |

*Brij 56 is cetyl alcohol POE (10)
Alfol 16RD is cetyl alcohol

The formulation below describes an emulsion cream suitable for administering an active component identified in the screening methods of the first aspect of the invention.

| Chemical Name or CTFA Name | Trade Name | Wt % | Wt % | Wt % |
|---|---|---|---|---|
| Coriander seed oil ex Loders | | 2.0 | 3 | 1.5 |
| Croklaan (PA triglyceride 60 - | | | | |
| 75% of total fatty acids) | | | | |
| Gallic acid | | 1 | 0 | 0 |
| Genistein | | 0 | 2 | |
| Diadzein | | 0 | 0 | 1.5 |
| Disodium EDTA | Sequesterene Na2 | 0.05 | 0.05 | 0.05 |
| Magnesium aluminium silicate | Veegum Ultra | 0.6 | 0.6 | 0.6 |
| Methyl paraben | Methyl Paraben | 0.15 | 0.15 | 0.15 |
| Simethicone | DC Antifoam Emulsion | 0.01 | 0.01 | 0.01 |
| Butylene glycol 1,3 | Butylene Glycol 1,3 | 3.0 | 3.0 | 3.0 |
| Hydroxyethylcellulose | Natrosol 250HHR | 0.5 | 0.5 | 0.5 |
| Glycerine, USP | Glycerine USP | 2.0 | 2.0 | 2.0 |
| Xanthan gum | Keltrol 1000 | 0.2 | 0.2 | 0.2 |
| Triethanolamine | Triethanolamine (99%) | 1.2 | 1.2 | 1.2 |
| Stearic acid | Pristerene 4911 | 3.0 | 3.0 | 3.0 |
| Propyl paraben NF | Propylparaben NF | 0.1 | 0.1 | 0.1 |
| Glyceryl hydrostearate | Naturechem GMHS | 1.5 | 1.5 | 1.5 |
| Stearyl alcohol | Lanette 18 DEO | 1.5 | 1.5 | 1.5 |
| Isostearyl palmitate | Protachem ISP | 6.0 | 6.0 | 6.0 |
| C12-15 alcohols octanoate | Hetester FAO | 3.0 | 3.0 | 3.0 |
| Dimethicone | Silicone Fluid 200 (50 cts) | 1.0 | 1.0 | 1.0 |
| Cholesterol NF | Cholesterol NF | 0.5 | 0.5 | 0.5 |
| Sorbitan stearate | Sorbitan Stearate | 1.0 | 1.0 | 1.0 |
| Butylated hydroxytoluene | Embanox BHT | 0.05 | 0.05 | 0.05 |
| Tocopheryl acetate | Vitamin E Acetate | 0.1 | 0.1 | 0.1 |
| PEG-100 stearate | Myrj 59 | 2.0 | 2.0 | 2.0 |
| Sodium stearoyl lactylate | Pationic SSL | 0.5 | 0.5 | 0.5 |
| Hydroxycaprylic acid | Hydroxycaprylic Acid | 0.1 | 0.1 | 0.1 |
| Alpha-bisabolol | Alpha-bisabolol | 0.2 | 0.2 | 0.2 |
| Water, DI | | to 100 | to 100 | to 100 |

Both of these topical compositions provide an effective cosmetic treatment to improve the appearance of wrinkled, aged, photodamaged, and/or irritated skin, when applied to normal skin that has deteriorated through the aging or photoageing or when applied to youthful skin to help prevent or delay such deteriorative changes. The compositions are also effective for soothing irritated skin, conditioning dry skin, lightening skin colour and reducing oil and sebum secretions. The compositions can be processed in conventional manner.

1. A method of identifying a compound that modulates NCKX-mediated calcium ion (Ca²⁺) exchange across a cell membrane, the method comprising the steps of:
(a) providing an adherent cell, the cell membrane of which comprises a NCKX polypeptide or a functionally equivalent variant thereof;
(b) exposing the cell in suspension to a fluorescent calcium-sensitive dye, thereby causing intracellular uptake of the dye; (c) settling the cell onto a solid support without allowing the cell to adhere to the solid support;
(d) exposing the cell to a test compound;
(e) exposing the cell to calcium ions (Ca²⁺) and potassium ions (K⁺); and (e) measuring fluorescence from the intracellular calcium-sensitive dye after exposure of the cell to the test compound and the calcium and potassium ions, thereby to determine the rate and/or amount of calcium ion exchange across the cell membrane in the presence of the test compound, wherein steps (a) and (b) are performed prior to steps (c) to (f).

2. A method according to claim 1 wherein step (c) is performed prior to steps (d) to (f).

3. A method according to claim 1 wherein steps (a) to (f) are performed in order.

4. A method according to claim 1 wherein steps (d) and (e) are performed substantially simultaneously.

5. A method according to claim 1, further comprising the step of measuring the fluorescence from the intracellular calcium-sensitive dye before exposure to the test compound and/or the calcium and potassium ions.

6. A method according to claim 1, further comprising the step of comparing the rate and/or amount of calcium ion exchange determined in step (f) in the presence of the test compound with a control measurement.

7. A method according to claim 6 further comprising the step of identifying whether the rate and/or amount of calcium ion exchange across the cell membrane has increased, decreased or stayed the same in response to exposure to the test compound in comparison to the control measurement.

8. A method according to claim 6 wherein the control measurement is obtained by performing steps (a), (b), (c), (e) and (f) in the absence of a test compound, thereby to determine the rate and/or amount of calcium ion exchange across the cell membrane in the absence of the test compound.

9. A method according to claim 6 wherein the control measurement is obtained by performing steps (b), (c), (d), (e) and (f) on a control cell, the cell membrane of which does not contain a NCKX polypeptide or a functionally equivalent variant thereof, thereby to determine the rate and/or amount of calcium ion exchange across the cell membrane in the absence of the NCKX polypeptide or the functionally equivalent variant thereof.

10. A method according to claim 6 wherein the control measurement is obtained by performing steps (a), (b), (c), (e) and (f) in the presence of a positive control compound known to stimulate NCKX-mediated calcium ion exchange across a cell membrane, thereby to determine the rate and/or amount of calcium ion exchange across the cell membrane in the presence of the positive control compound.

11. A method according to claim 6 wherein the control measurement is obtained by performing steps (a), (b), (c), (e) and (f) in the presence of a negative control compound known to inhibit NCKX-mediated calcium ion exchange across a cell membrane, thereby to determine the rate and/or amount of calcium ion exchange across the cell membrane in the presence of the negative control compound.

12. A method according to claim 6 wherein the control measurement is obtained by performing steps (a) to (f) in the presence of a selective blocker of voltage-gated Ca²⁺ channels, a selective blocker of receptor-operated Ca²⁺ channels, a selective blocker of the Na⁺ pump, and/or a selective blocker of NCX-type ion exchangers, thereby to determine whether the test compound is acting via the NCKX-polypeptide.

13. A method according to claim 1, wherein prior to step (b), the adherent cell is trypsinised, thereby to bring the cell into suspension.

14. A method according to claim 1, wherein the solid support comprises a cell culture chamber or well.

15. A method according to claim 14 wherein the cell culture chamber or well has been treated to facilitate cell binding.

16. A method according to claim 15 wherein the cell culture chamber or well has a polystyrene resin coating.

17. A method according to claim 1, wherein the cell is a Hamster Embryonic Kidney (HEK) cell, a High Five™ insect cell, a dictyostelium cell, a tobacco plant cell or a p53 deficient cell line H 1299 cell.

18. A method according to claim 1, wherein the NCKX polypeptide is NCKX2, or a functionally equivalent variant thereof.

19. A method according to claim 1, wherein the NCKX polypeptide is NCKX5, or a functionally equivalent variant thereof.

20. A method according to claim 1, wherein the NCKX polypeptide is naturally located in the cell membrane.

21. A method according to claim 1, wherein the NCKX polypeptide, or functionally equivalent variant thereof, has been recombinant Iy targeted to the cell membrane.

22. A method according to claim 21 wherein the NCKX polypeptide is targeted to the membrane by linking the NCKX polypeptide to a leader sequence and/or tag that targets polypeptides to, and for inclusion in, a cell membrane.

23. A method according to claim 22 wherein the leader sequence is derived from NCKX2, NCKX4, yeast a mating factor, an NCX protein, TGFbeta, haemagglutinin or a viral surface protein.

24. A method according to claim 23 wherein the leader sequence comprises the N terminal sequence of Homo sapiens NCKX2 (hNCKX2) (amino acids 1 to 120).

25. A method according to claim 1, wherein the NCKX polypeptide or functionally equivalent variant possesses the amino acid residue Thr at the position equivalent to residue 111 of NCKX5.

26. A method according to claim 1, wherein the NCKX polypeptide or functionally equivalent variant possesses the amino acid residue Ala at the position equivalent to residue 111 of NCKX5.

27. A method according to claim 1, wherein the test compound is selected from a peptide, a nucleic acid molecule, a small organic molecule, a small inorganic molecule, a natural compound, an extract of a natural compound, a mixture of natural compounds or extracts thereof, and combinations thereof.

28. A method according to claim 1, wherein step (d) comprises simultaneously exposing the cell to a plurality of test compounds, and the method further comprises the step of isolating and/or identifying individual test compounds from the plurality of test compounds.

29. A method according to claim 28 further comprising the step of individually retesting the isolated and/or identified test compounds for the ability to modulate NCKX-mediated calcium ion exchange across a cell membrane.

30. A method according to claim 1, further comprising the step of identifying a test compound that is found to have the ability to modulate NCKX-mediated calcium ion exchange across the cell membrane.

31. A method according to claim 30 wherein the identified compound is modified, and the modified compound is tested for the ability to modulate NCKX-mediated calcium ion exchange across a cell membrane.

32. A method according to claims 30 wherein the identified compound or the modified compound is tested for toxicity in a cellular, tissue or animal model of toxicity.

33. A method according to claim 30, wherein the identified compound or the modified compound is tested for the ability to modulate calcium ion exchange, mediated by a desired NCKX polypeptide, across a cell membrane.

34. A method according to claim 30 wherein the identified compound or the modified compound is tested for the ability to selectively modulate calcium ion exchange, mediated by a desired NCKX polypeptide, across a cell membrane.

35. A method according to claim 33 wherein the desired NCKX polypeptide is NCKX5.

36. A method according to claim 35, wherein the identified compound or the modified compound is further tested for the ability to modulate skin pigmentation.

37. A method according to claim 35, wherein the identified compound or the modified compound is further tested for the ability to modulate hair pigmentation.

38. A method according to claim 35, wherein the identified compound or the modified compound is further tested for the ability to modulate retinal pigmentation.

39. A method according to claim 30, wherein a compound which increases the rate and/or amount of NCKX5-mediated calcium ion exchange is tested for efficacy in a model of a disease or condition selected from sun-induced skin damage, skin cancer, a disease characterised by vitamin D deficiency such as osteoporosis, and a disease or condition characterised by sensitivity to UV and/or visible light such as porphyria and xeroderma pigmentosum.

40. A method according to claim 30, wherein a compound which increases the rate and/or amount of NCKX5-mediated calcium ion exchange is tested for efficacy as a tanning agent

41. A method according to claim 30, wherein a compound which decreases the rate and/or amount of NCKX5-mediated calcium ion exchange is tested for efficacy in a model of disease or condition characterised by elevated or excessive pigmentation such as melanoma, melasma and increased pigmentation through inflammation.

42. A method according to claim 30, wherein a compound which decreases the rate and/or amount of NCKX5-mediated calcium ion exchange is tested for efficacy in a model of skin, hair or retinal lightening.

43. A method according to claim 42 wherein the compound is tested for efficacy in a model of age spots or mottled skin pigmentation.

44. A method according to claim 30, wherein a compound which modulates the rate and/or amount of NCKX5-mediated calcium ion exchange is tested for the ability to modulate coat or skin colour in an animal.

45. A method according to claim 30, wherein a compound which modulates the rate and/or amount of NCKX1-, NCKX2- or NCKX5-mediated calcium ion exchange is tested for efficacy in a model of a retinal disease or condition.

46. A method according to claim 45 wherein the retinal disease is age-related macular degeneration.

47. A method according to claim 30, wherein a compound which modulates the rate and/or amount of NCKX2-mediated calcium ion exchange is tested for efficacy in an animal model of memory or learning

48. A method according to claim 30, wherein a compound which modulates the rate and/or amount of NCKX2-mediated calcium ion exchange is tested for efficacy in a model of a neurodegenerative disease.

49. A method according to claim 48 wherein the neurodegenerative disease is Alzheimer's disease, Parkinson's disease or Huntington's disease.

50. A method according to claim 30, wherein a compound which modulates the rate and/or amount of NCKX3- or NCKX4-mediated calcium ion exchange is tested for efficacy in model of a vascular smooth muscle cell disorder.

51. A method according to claim 50 wherein the disorder of vascular smooth muscle cells is selected from hypertension, atherosclerosis, restenosis, diabetes, renal pathologies, asthma, obstructive bladder disease, and various gastrointestinal and reproductive disorders, and solid tumours.

52. A method according to claim 30, further comprising the step of synthesising and/or purifying the identified compound or the modified compound.

53. A method according to claim 30, further comprising the step of formulating the identified compound or the modified compound into a cosmetic or pharmaceutical formulation.

54-55. (canceled)
